(19) United States
(12) Patent Application Publication
(10) Pub. No.: US 2007/0065922 A1
(43) Pub. Date: Mar. 22, 2007

(54) Biocatalytic synthesis of aminodeoxy purine N9-beta-D-nucleosides containing 3-amino-3-deoxy-beta-D-ribofuranose, 3-amino-2,3-dideoxy-beta-D-ribofuranose, and 2-amino-2-deoxy-beta-D-ribofuranose as sugar moieties

(75) Inventors: Vladimir N. Barai, Kuopio (FI); Ludmilla A. Eroshevskaya, Kuopio (FI); Igor A. Mikhailopulo, Kuopio (FI); Alex V. Azhayev, Siilinjärvi (FI); Seppo Perva Lapinjoki, Heinola (FI)

Correspondence Address: BANNER & WITCOFF, 1001 G STREET N W, SUITE 1100, WASHINGTON, DC, 20001, US

(73) Assignee: Metkinen Oy, Kuusisto (FI)

(21) Appl. No.: 11/288,414

(22) Filed: Nov. 29, 2005

Related U.S. Application Data
(60) Provisional application No. 60/718,722, filed on Sep. 21, 2005.

(51) Int. Cl. C12P 19/30; C07H 19/16; C07H 19/19

(52) U.S. Cl. 435/89; 536/27.3; 536/27.4

(57) ABSTRACT

Purine N⁹-β-D-nucleosides containing 3-amino-3-deoxy-β-D-ribofaranose, 3-amino-2,3-dideoxy-β-D-ribofuranose, and 2-amino-2-deoxy-β-D-ribofuranose as sugar moieties are synthesized by biocatalytic transglycosylation of purine bases and the respective 3′-amino-3′-deoxyuridine, 3′-amino-3′-deoxythymidine and 2′-amino-2′-deoxyuridine as donors of the carbohydrate moiety, and the cells of Escherichia coli as a biocatalyst or glutaraldehyde (GA) treated cells of Escherichia coli as a biocatalyst or a mixture of thymidine (uridine) phosphorylase and purine nucleoside phosphorylase.

This application claims priority to provisional application U.S. Ser. No. 60/718,722, filed Sep. 21, 2005, which is hereby incorporated by reference in its entirety.

### FIELD OF THE INVENTION

The present invention relates to synthesis of aminodeoxy purine N⁹-β-D-nucleosides containing 3-amino-3-deoxy-β-D-ribofuranose, 3-amino-2,3-dideoxy-β-D-ribofuranose, and 2-amino-2-deoxy-β-D-ribofuranose as sugar moieties by biocatalytic transglycosylation.

### BACKGROUND OF THE INVENTION

Aminodeoxy nucleosides represent a group of nucleoside antibiotics with a broad spectrum of biological activities [Suhadolnik, R. J. Nucleoside Antibiotics; Wiley: New York, 1970; 1-50; Suhadolnik, R. J. Nucleosides as Biological Probes; Wiley: New York, 1979; 96-102.] Moreover, nucleosides containing an amino group at the 2′- or 3′-position have valuable potential for the investigation of chemical and/or biochemical problems, in which the ribofuranose moiety is involved (e.g., [Krider, E. S. et al., Bioconjugate Chem. 2002, 13, 155-162; Ford, L. P. et al. J. Biol. Chem. 2001, 276, 32198-32203.]).

During the last decade, the oligonucleotide N3′→P5′ phosphoramidate attracted much attention owing to its very interesting physico-chemical and biological properties [Gryaznov, S. M. Oligonucleotide N3-->P5′ phosphoramidates as potential therapeutic agents. Biochim. Biophys. Acta. 1999, 1489, 131-40; Shea-Herbert, B. et al., Oncogene 2002, 21, 638-642.] These types of oligonucleotides form very stable duplexes with complimentary native phosphodiester DNA and exceptionally stable duplexes with RNA strands. Moreover, the phosphoramidate compounds form extremely stable triple stranded complexes with single or double stranded DNA oligomers under near physiological salt and pH conditions. They are resistant to enzymatic digestion by nucleases both in vitro and in vivo. Oligonucleotide phosphoramidates apparently are cell permeable, and they have a good bioavailability and biodistribution, while being non-toxic at therapeutically relevant doses. Finally, oligonucleotide phosphoramidates are efficient telomerase inhibitors. Human telomerase is a unique reverse transcriptase that is expressed in multiple cancers, but not in the vast majority of normal cells. It has been proposed that the specific inhibition of telomerase activity in tumors might have significant beneficial therapeutic effects [JP 2003012688; U.S. Pat. No. 6,169,170; U.S. Pat. No. 5,965,720; AU 704,549; PL 328,639; U.S. Pat. No. 5,859,233; EPO 882,059; U.S. Pat. No. 5,837,835; U.S. Pat. No. 5,726,297; U.S. Pat. No. 5,684,143; AU 6,178,996; WO 9,737,691; HU 76,094; CZ 9,602,745; U.S. Pat. No. 5,631,135;U.S. Pat. No. 5,599,922; U.S. Pat. No. 5,591,607; WO 9,525,814; EPO 754,242; AU 2,190,095].

A central problem of using aminodeoxy nucleosides for the preparation of medicinal drugs, biochemical tools, components of diagnostic means, or oligonucleotide phosphoramidates is the availability of the parent aminodeoxy nucleosides. There are numerous publications that address this problem.

Considerable efforts had been devoted to the chemical synthesis of aminodeoxy nucleosides. There are two major strategies for the synthesis of sugar-modified nucleosides: (i) chemical modification of the now readily available natural nucleosides, and (ii) coupling of appropriately modified glycosyl donors with heterocyclic bases.

### 2-Amino-2-deoxy-β-D-ribofuranosyl nucleosides

Chemical modification of the natural nucleosides. From a chemical viewpoint, two different approaches should be considered, viz., synthesis of aminodeoxy nucleosides of pyrimidines and purines. The former can be easily prepared in many cases through intermediate formation of O²,2′-anhydro derivatives of uridine and thymidine, respectively. Since the first observation of the formation of O²,2′-anhydro derivatives of pyrimidine nucleosides 2 and 6 from, e.g., 5′-O-acetyl-2′-O-tosyluridine (1) [Brown, D. M. et al. J. Chem. Soc. 1957, 868; Brown, D. M. et al., J. Chem. Soc. 1958, 4242] or 1-(5-O-trityl-2-O-mesyl-β-D-ribofuanosyl)thymine (5) [Fox, J. J et al. J. Am. Chem. Soc. 1957, 79, 2775-2778], on treatment with bases, a number of different methods was suggested for this transformation. Todd et al. have not observed the formation of the expected 5′-O-acetyl-2′-azido-2′-deoxyuridine (3) that was explained by the low solubility of the sodium azide in acetonitrile [supra, J. Chem. Soc. 1957]. Direct transformation of uridine into O²,2′-anhydrouridine under the action of diphenyl carbonate in DMF originally described by Hampton and Nichol [Hampton, A. et al. Biochemistry 1966, 5, 2076] remains the most efficient method and it was recently used for the synthesis on the kilogram scale in 75-85% yield [Verheyden, J. P. H. et al. J. Org. Chem. 1971, 36, 250-254]. It is noteworthy that pyrimidine O²,2′-anhydro nucleosides are valuable sources of the corresponding β-D-arabinofuranosyl nucleosides as it was first stated by Fox et al. by the synthesis of 1-(β-D-arabinofuranosyl)-thymine (ara-T; 8) [supra, J. Am. Chem. Soc. 1957, 79].

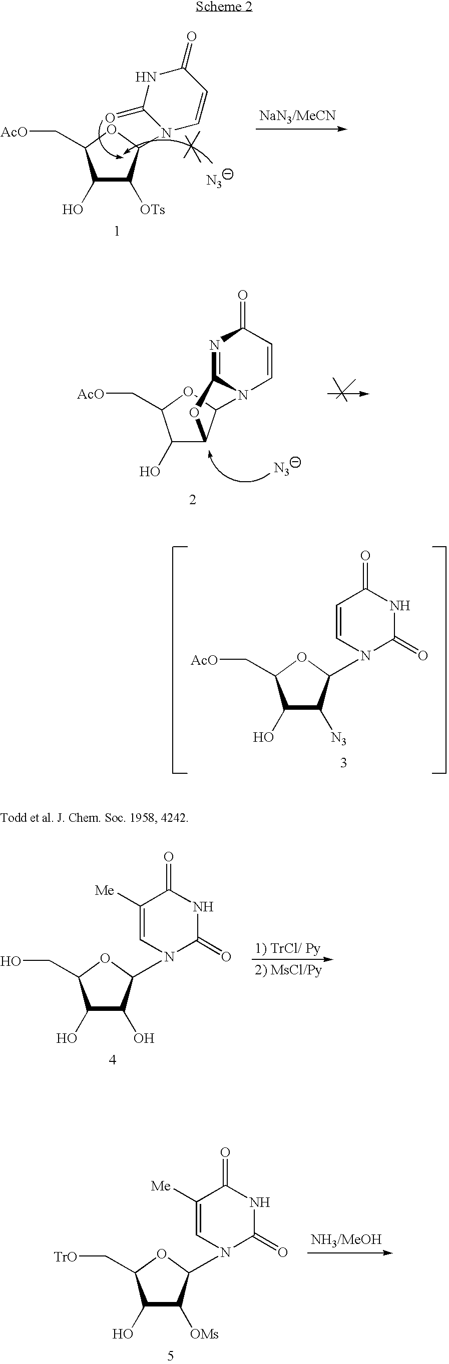

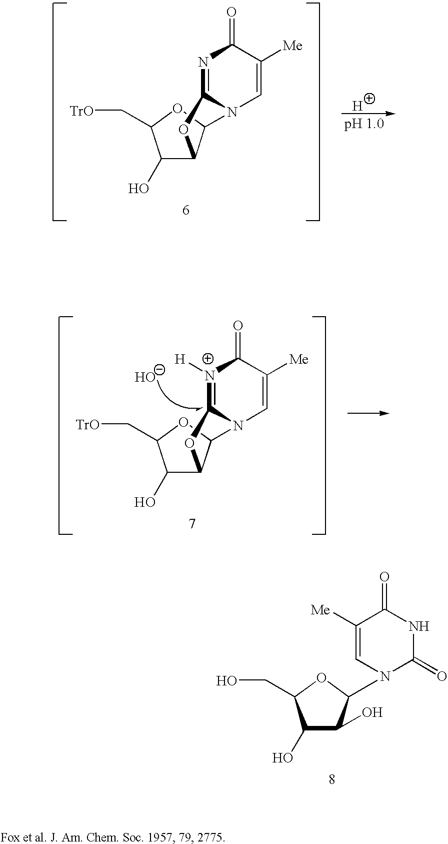

First transformation of O²,2′-anhydrouridine (10) into 2′-azido-2′-deoxyuridine (11) and then to 2′-amino-2′-deoxyuridine (12) was described in 1971 by Moffatt et al. [supra J. Org. Chem. 1971, 36]. As distinct from Todd's works [supra, J. Chem. Soc. 1957, supra, J. Chem. Soc. 1958], more soluble LiN₃ in hexamethylphosphoramide (HMPT) was employed for the O²,2′-anhydro-ring opening under very drastic conditions (150° C.) to afford, after silicic acid column chromatography, the desired azide 11 in 50% yield. Catalytic reduction of the latter gave 12 in 98% yield (Scheme 3). Synthesis of 2′-amino-2′-deoxycytidine (15) was also accomplished by the general thiation-amination procedure of Fox et al. [J. Am. Chem. Soc. 1959, 81, 178] (Scheme 3).

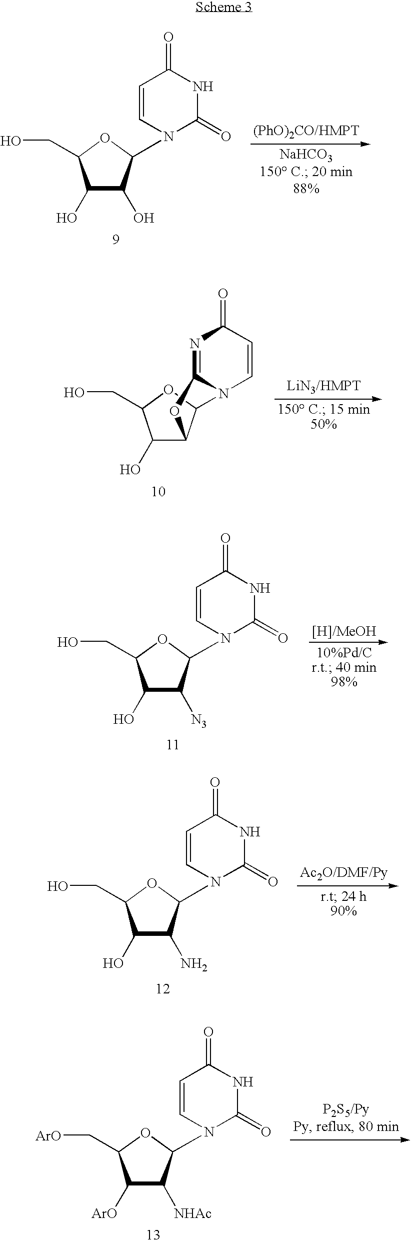

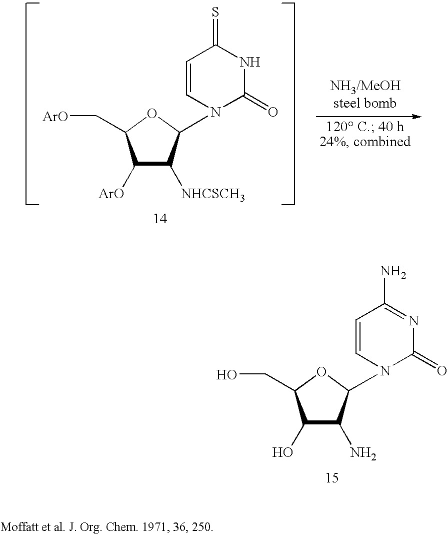

During the following years, the method by Moffatt et al. was essentially improved at the first stage employing minimal volume of DMF as a solvent and conducting the reaction at 110-120° C. for 4-5 h affording 10 in 84% yield without chromatography [McGee, D. P. C. et al. J. Org. Chem. 1966, 61, 781-785], and at the reduction of an azido group to amino by the use of triphenylphosphine (Staudinger reaction; 91% yield) [Imazawa, M. et al. J. Org. Chem. 1979, 44, 2039-2041]. The transformation of uracil nucleosides, e.g., 3′,5′-di-O-protected azide 11, to the corresponding cytosine nucleosides may be realized by the very efficient two step procedure (1—POCl₃/1,2,4-triazole/NEt₃; in anh MeCN; 2—NH₄OH in dioxane [Divakar, K. J. et al. J. Chem. Soc., Perkin Trans. 1 1982, 179-183]). Despite the aforementioned improvements, the critical stage—the O²,2′-anhydro-ring opening by nucleophilic agents remained rather laborious and low yielding procedure precluding from the preparation of 12 and 15 according this route on preparative level.

Recently, two new syntheses of both pyrimidine nucleosides 12 and 15 have been developed. First of them uses O²,2′-anhydrouridine (10) as starting nucleoside [supra J. Org. Chem. 1966, 61]. It is protected at the 5′-hydroxyl group (16) and then treated with trichloroacetonitrile (CCl₃CN) in the presence of catalytic amount of and NEt₃ under reflux to afford the oxazoline 17 in 70-80% isolated yield [supra J. Org. Chem. 1966, 61]. The one-pot conversion of O²,2′-anhydrouridine (10) to oxazoline 17, without intermediate isolation of 16, was accomplished with similar efficiency. Oxazoline 17 was shown to be valuable versatile intermediate. Its treatment with 80% AcOH led to the formation of 2′-amino-2′-deoxyuridine (12) in 80% yield. Alternatively, its treatment with strong ethanolic sodium hydroxide gave 5′-ODMT-protected derivative of 2′-amino-2′-deoxyuridine (70-80%), which was used for the preparation of structural block for automated oligonucleotide synthesis [supra J. Org. Chem. 1966, 61]. Moreover, oxazoline 17 was readily transformed to 2′-amino-2′-deoxycytosine (15) (ca. 50%; combined) (Scheme 4). This efficient and novel approach to the synthesis of 2′-amino-2′-deoxy-uridine (12) and -cytosine (15) makes these nucleosides readily available for diverse applications.

The second recently published approach was exemplified by the synthesis of 2′-amino-2′-deoxy-uridine (12), -cytosine (15) and -adenosine (32) as well as the phosphoamidite building blocks for oligonucleotide synthesis (not shown) using the respective β-D-arabinofuranosyl nucleosides as starting compounds [Karpeisky, A. et al. Bioorg. Med. Chem. Letters 2002, 12, 3345-3347] (Scheme 5).

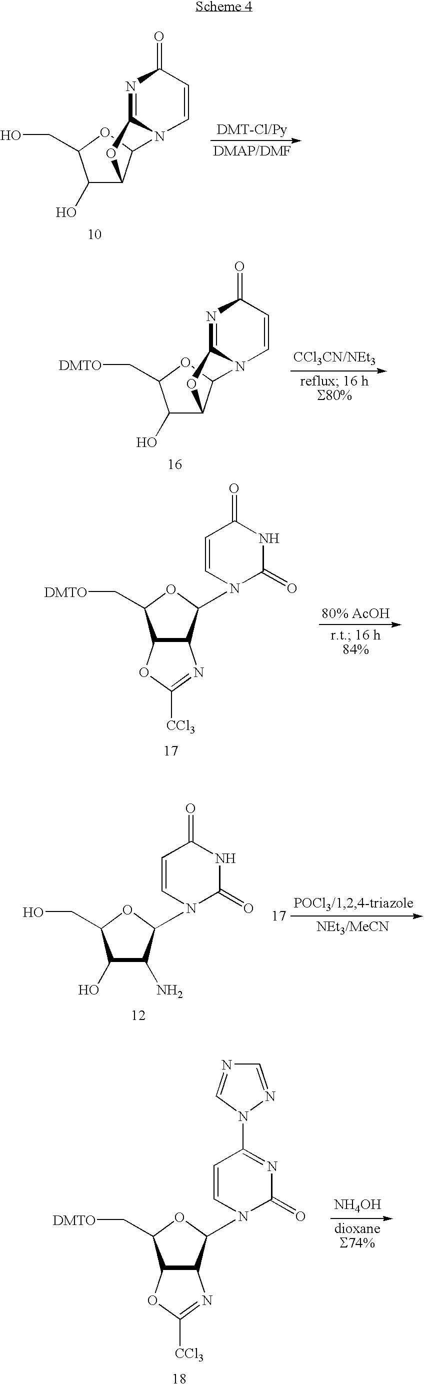

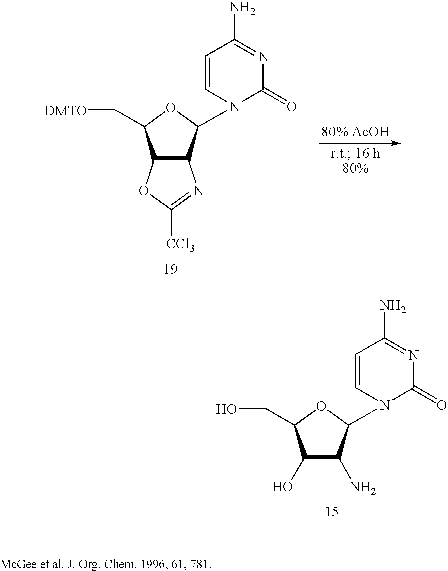

The use of the rather expensive tetraisopropyldisiloxyl group (Markiewicz group) for the simultaneous protection of 3′- and 5′-hydroxyls of the starting arabinosides 20-22 is a serious drawback of this scheme. Moreover, arabinofuranosides of purines as distinct from the pyrimidine counterparts are not readily available and cheap starting compounds and their synthesis represents independent challenge.

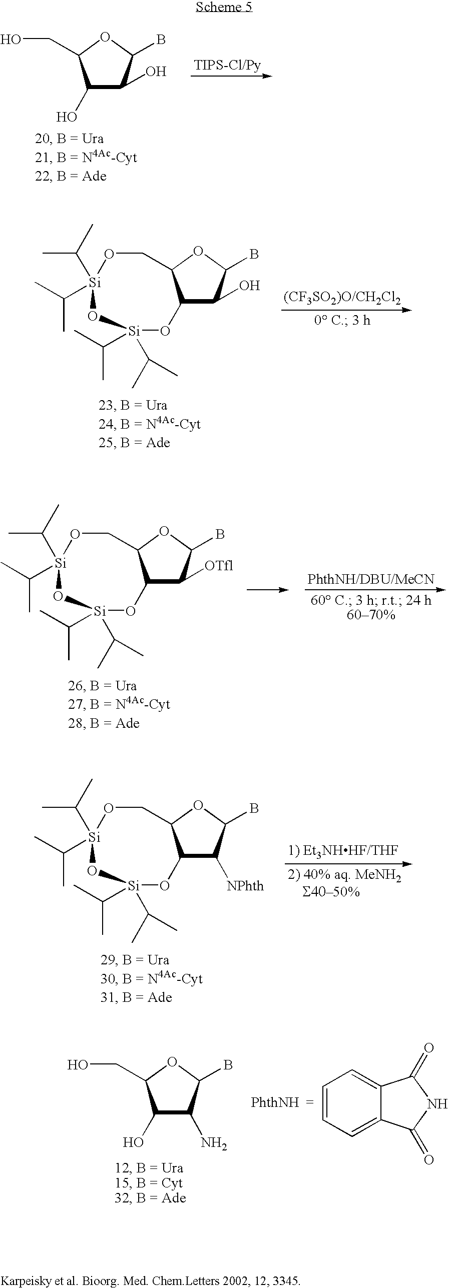

Synthesis of 2′-amino-2′-deoxyadenosine (32) similar to that by Karpeisky et al. was previously described by M. J. Robins et al. [Nucleosides Nucleotides 1992, 11, 821-834]. Treatment of triflate 28 with LiN₃ in DMF (r.t.; 2 h), followed by removal of TPDS group [Bu₄NF/THF; r.t.; 16 h; Dowex 1×2 (OH⁻-form; MeOH—H₂O elution] and reduction of the azido group to amino [1—Staudinger conditions: Ph₃P in pyridine and saturated (0° C.) NH₃/MeOH (1:1, vol); Dowex 1×2 (OH⁻-form; H₂O elution); 44%; 2-Bu₃SnH/AIBN [Poopeiko, N. E. et al. SynLett 1991, 342]; DMAC-benzene (1:3, vol), reflux, 1 h; Dowex 1×2 (OH⁻-form; H₂O elution; 78%] gave 2′-amino-2′-deoxyadenosine (32) in 18-33% combined yield. It should be stressed that the all methods based on the use of natural purine nucleosides as starting compounds (cf. [Mengel, R. et al. Chem. Ber. 1976, 109, 433-443; Ikehara, M. et al. Chem. Pharm. Bull. 1978, 26, 240; Ranganathan, R. Tetrahedron Lett. 1977, 18, 1291]) are rather lengthy and laborious, and can be hardly employed for the synthesis of purine 2-amino-2-deoxy-β-D-ribofuranosyl nucleosides on the preparative scale.

Coupling of purine bases with suitable amino sugars or their precursors. The first synthesis of 2′-amino-2′-deoxyadenosine (32) by the condensation of base with sugar was described by Wolfrom and Winkley in 1967 [J. Org. Chem. 1967, 32, 1823-1825] (Scheme 6). Starting sugar, ethyl 2-deoxy-2-(2,4-dinitroanilino)-1-thio-α-D-ribofuranoside (33), was prepared in many steps from 2-amino-2-deoxy-D-glucose. Conventional acetylation of 33 followed by the treatment of the resulted 34 with chlorine in CH₂Cl₂ gave oily chloride 35, which was immediately brought into reaction with 6-acetamido-9-chloromercuripurine (36) in refluxing toluene. Tedious work-up of the reaction mixture, followed by removal of protecting groups afforded a 1:1 anomeric mixture of nucleosides, which was separated into individual β-D-(32) and α-D-anomers (37).

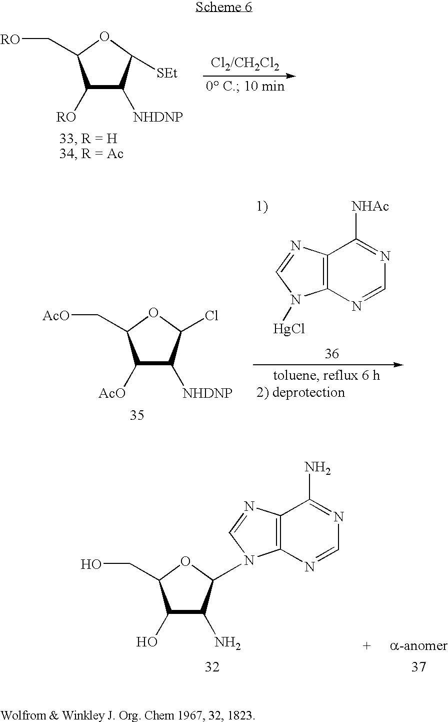

Later, Hobbs and Eckstein described the synthesis of 2′-amino-2′-deoxy-adenosine (32) and -guanosine (50) using 1,3,5-tri-O-acetil-2-azido-2-deoxy-D-ribofuranose (41) as universal glycosylating agent [J. Org. Chem. 1977, 42, 714-719]. Synthesis of the latter was realized from uridine (9) in 6 steps through intermediate consecutive formation of O²,2′-anhydrouridine (10), 2′-azido-2′-deoxyuridine (11) (acc. to Moffatt et al. supra, J. Org. Chem 1971, 36), 2-azido-2-deoxy-D-ribose (38), methyl 2-azido-2-deoxy-D-ribofuranoside (39), its peracyl derivative 40, and, finally, acetate 41 in a 18% combined yield. Condensation of acetate 41 with N⁶-octanoyladenine (42) in the presence of SnCl₄ in 1,2-dichloromethane gave, after work-up, deprotection and Dekker chromatography 2′-azido-2′-deoxyadenosine (43) and its N⁹-α-isomer 44 in a ratio of 2:1 (59%; combined). Similar condensation of acetate 41 with N²-palmitoylguanine (45) gave more complicated reaction mixture, from which N⁹-O-anomer 46 and N⁷-β-anomer 48 were isolated in 15 and 21% yield, respectively. Staudinger reduction of the azides 43, 46 and 48 gave the respective 2′-amino-2′-deoxy-β-D-ribofuranosyl adenine 32 and guanine 50 and 51 nucleosides in high yield (Scheme 7). This synthetic method affords 2′-amino-2′-deoxy-adenosine (32) and -guanosine (50) in 9 and ca. 1% total yield, respectively, based on uridine as starting material. Advantage of this method consists in the use of the universal glycosylating agent 41.

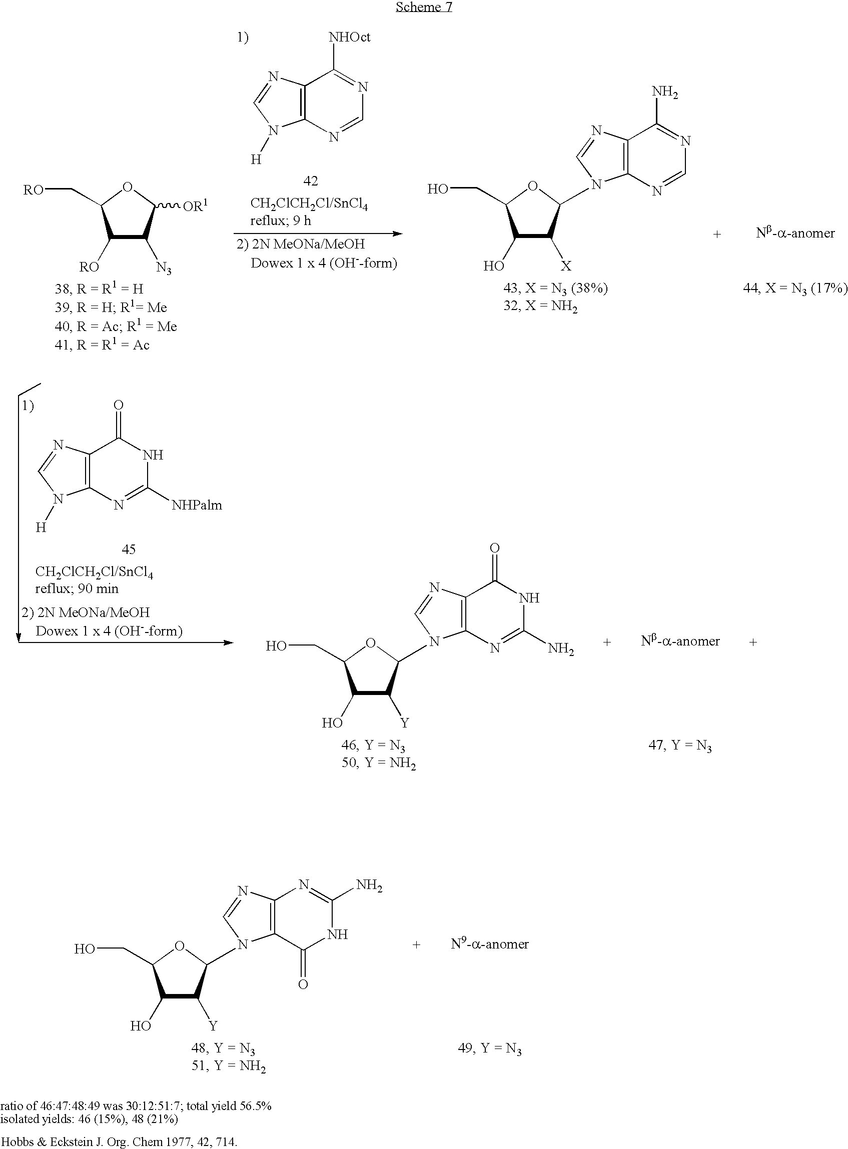

Shortly after this publication, an improvement of this method was developed by Imazawa and Eckstein using 2′-deoxy-2′-trifluoroacetamidouridine (52) as a donor of the pentofuranose moiety in the reaction of chemical transglycosylation of purine bases 42 and 45 [J. Org. Chem. 1979, 44, 2039-2041]. 2′-Amino-2′-deoxyuridine (12) was transformed to 52 by the treatment with S-ethyl trifluorothioacetate in 88% yield. The latter was used for an one-pot silylation-transglycosylation reaction with N⁶-octanoyladenine (42) in the presence of trimethylsilyl trifluoromethanesulfonate (TMS-Tfl) in acetonitrile to afford, after work-up, deprotection, and Dekker chromatography and finally Dowex 50W×8 (NH₄⁺-form) chromatography 2′-amino-2′-deoxyadenosine (32) (34%) and its N⁹-α-isomer (53) (7%). In a similar way, replacement of 42 by N²-palmitoylguanine (45) in the above reaction gave 2′-amino-2′-deoxyguanosine (50) in a 60% yield. Unexpectedly, neither N⁹-α-isomer of 50 nor N′-isomers were detected in the reaction mixture (Scheme 8).

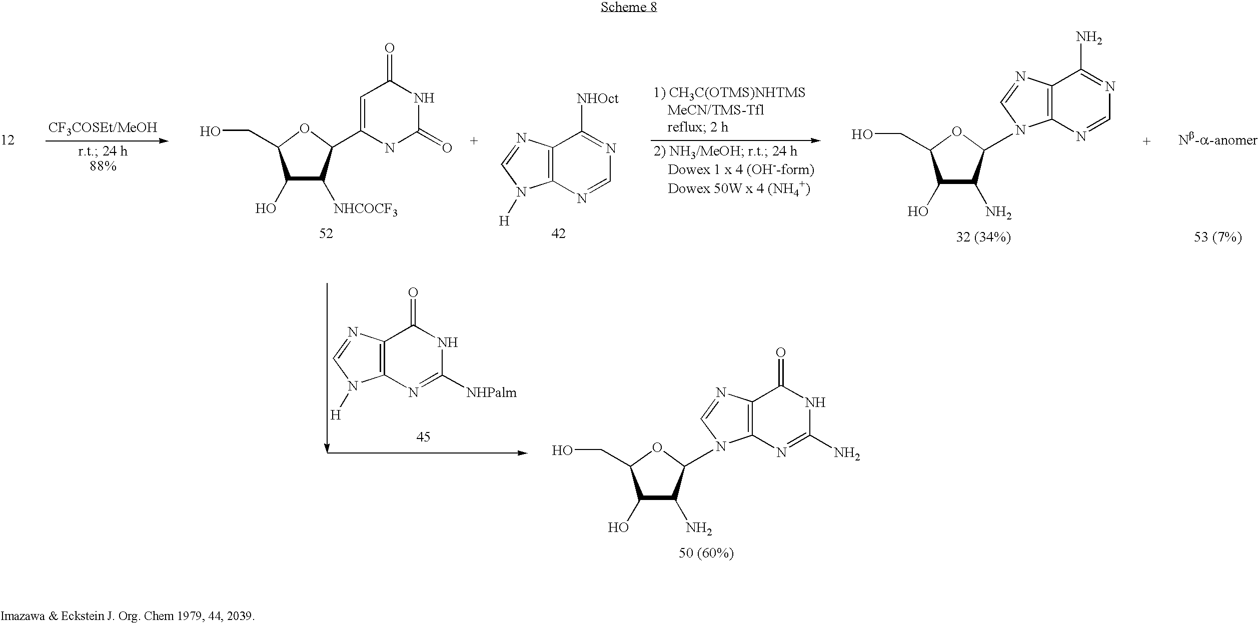

This latter method of the synthesis of purine 2-amino-2-deoxy-β-D-ribonucleosides remains at present the best chemical route to this group of modified nucleosides.

### 3-Amino-3-deoxy-β-D-ribofuranosyl nucleosides

Chemical modification of the natural nucleosides. The isolation of nucleoside antibiotic puromycin and discovery of its activity in a broad range of organisms and experimental tumors attracted vast attention of researchers to this class of compounds [1]. It was shown that 6-dimethylamino-9-(3-amino-3-deoxy-β-D-ribofuranosyl)purine is the biologically active nucleoside moiety of puromycin that stimulated extensive chemical studies directed towards the preparation of this nucleoside and its diverse analogues. All efforts were concentrated on the research and development of convergent approach, viz., condensation of suitable carbohydrate derivative with heterocyclic bases. Interestingly, an early attempt to use of O²,3′-anhydro pirimidine derivatives for the introduction of amino function into nucleoside had failed; it was unexpectedly found that 3′-O-tosyluridine is extremely resistant to both inter- and intramolecular nucleophilic attack [Brown, D. M. et al. J. Chem. Soc. 1958, 3028.].

The first synthesis of 3′-amino-3′deoxyadenosine (32) from adenosine through intermediate formation of 9-(2,3-anhydro-β-D-lyxofuranosyl)adenine, its transformation to 9-(3-azido-3-deoxy-β-D-xylofuzanosyl)adenine on treatment with LiN₃, followed by inversion of configuration at C2′ and finally reduction of azido group to amino was very lengthy (12 steps) and laborious (ca. 3% overall yield) [Supra, Chem. Ber. 1976, 109].

Two more efficient routes have been elaborated by M. J. Robins et al. [supra, Nucleosides Nucleotides 1992, 11; J. Org. Chem. 2001, 66, 8204-8210]. One of them used readily available 3′,5′-di-O-(t-butyldimethylsilyl)adenosine (54) as starting compound. Stereoselective inversion of configuration at C3′ by oxidation/reduction procedure gave xyloside 55, hydroxyl group of which was activated by the formation of trifluoromethanesulfonate (triflate; Tfl) ester 56. The 3′-xylo-triflyloxy group underwent substitution with inversion of configuration under very mild conditions. The crude 3′-ribo-azido derivative was deprotected (Bu4N⁺F⁻/THF) and finally the azido group was reduced to amino to afford the desired 3′-amino-3′deoxyadenosine (32) in ca. 30% total yield (Scheme 9).

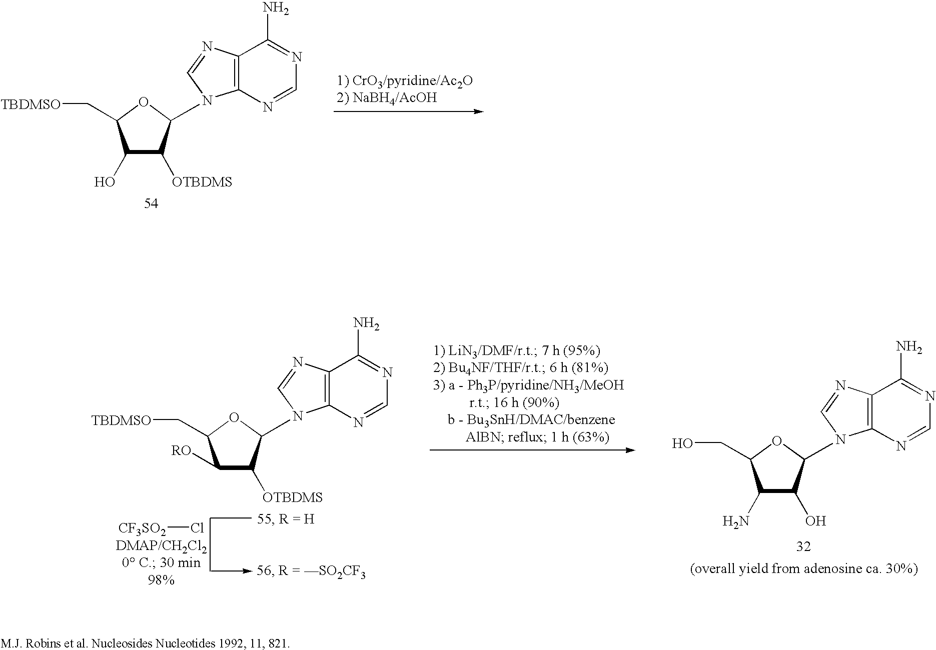

This method was recently employed for general synthesis of puromycin analogues [Nguyen-Trung, N. Q. et al. J. Org. Chem. 2003, 68, 2038-2041].

The other route consists of 9-stage synthesis and starts with an one-pot transformation of adenosine (58) to 9-(2,3-anhydro-β-D-ribofuranosyl)adenine (59). Protection of the 5′-hydroxyl group with t-butyldiphenylsilyl (TBDPS) group followed by the treatment with dimethylboron bromide led to bromoxyloside (60). The latter was treated with benzylisocyanate to afford the 2′-O-(N-benzylcarbamoyl) derivative 61, which underwent ring closure with inversion of configuration at C3′ on reaction with NaH, and the resulted oxazolidinone was desilylated to give 62. Consecutive treatment of 62 with aq. NaOH and N-debenzylation using Perlman's catalyst [Pd(OH)₂—C/HCOO⁻NH₄⁺] gave 57 in 56% overall yield (Scheme 10).

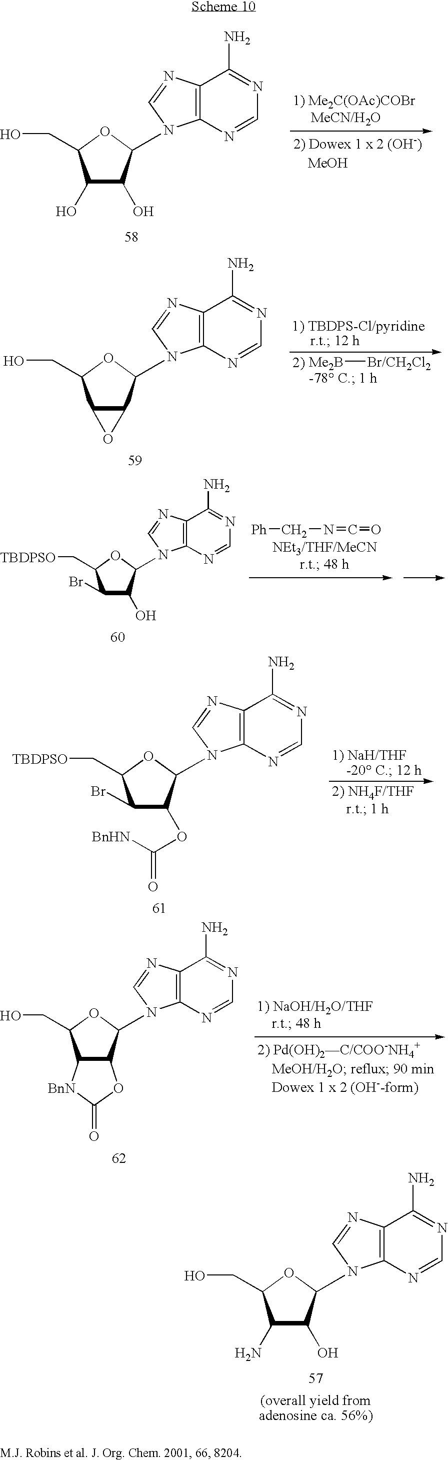

Based on this method, a practical approach to 3′-amino-3′-deoxyadenosine and puromycin analogues was elaborated [Supra. J. Org. Chem. 2003, 68].

Recently, this synthetic route was improved and used for the preparation of both 3′-amino-3′deoxyadenosine (57) and 3′-amino-3′deoxyguanosine (67) [Zhang, L. et al. Helv. Chim Acta 2003, 86, 703-710] (Scheme 11). It is noteworthy that it is the first synthesis of 3′-amino-3′deoxyguanosine (67) from guanosine (63) and some interesting features should be noticed. First, guanosine (63) was transformed in three steps into bromide 64, careful deacetylation of which gave xyloside 65 in high yield. Second, xyloside 65 was consecutively treated with benzylisocyanate and then with NaH in DMF under more vigorous conditions as described in [supra, J. Org. Chem. 2001, 66], which resulted in oxazolidinone ring closure accompanied with the removal of TBDPS protecting group. Finally, treatment of 66 with 3.0N NaOH in aq. MeOH followed by N-debenzylation using Perlman's catalyst in a mixture of EtOH—AcOH at room temperature gave 67 in 58% overall yield.

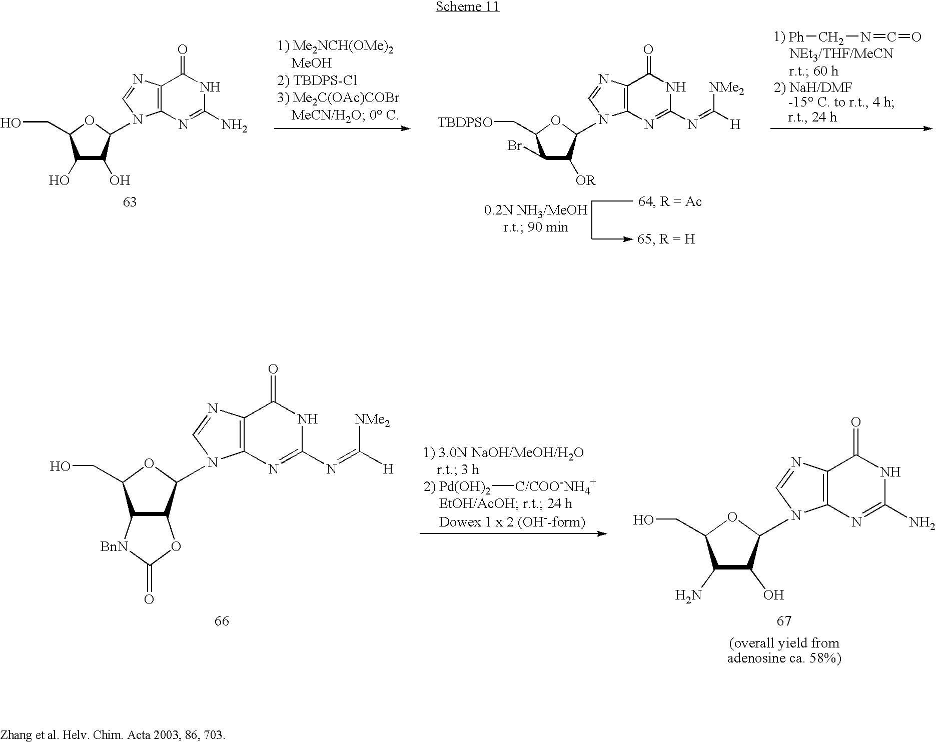

Coupling of purine bases with suitable amino sugars or their precursors. The first convergent synthesis of 6-dimethylamino-9-(3-amino-3-deoxy-β-D-ribofuranosyl)purine (79) [Baker, B. R. et al. J. Am. Chem. Soc. 1955, 77, 7-12. Baker, B. R. et al. J. Am. Chem. Soc. 1955, 77, 12-15] and 3′-amino-3′-deoxyadenosine (57) [Baker, B. R. et al. J. Am. Chem. Soc. 1955, 77, 5911-5915.] by the condensation of heterocyclic base with sugar was described by Baker and co-workers in 1955 (Scheme 12). Synthesis of puromycine nucleoside 79 consisted of 18 steps starting from D-xylose (68) and gave the desired product in ca. 0.3% total yield. The following important general features of this route should be noted. First, transformation of the readily available D-xylose (68) to α- and β-methyl xylosides 69 and 70, respectively, was repeatedly used since this work for the preparation of diverse sugar derivatives, which have then been used as universal glycosylating agents. Second, neighboring group participation for an inversion configuration at carbon atom of pentofuranose ring, viz., a 72→74 transformation, had found numerous applications. Third, the use of TiCl₄—a catalyst of Friedel-Crafts alkylation's of aromatic compounds for the coupling of heterocyclic base and sugar was later developed to the general method of convergent synthesis of nucleosides.

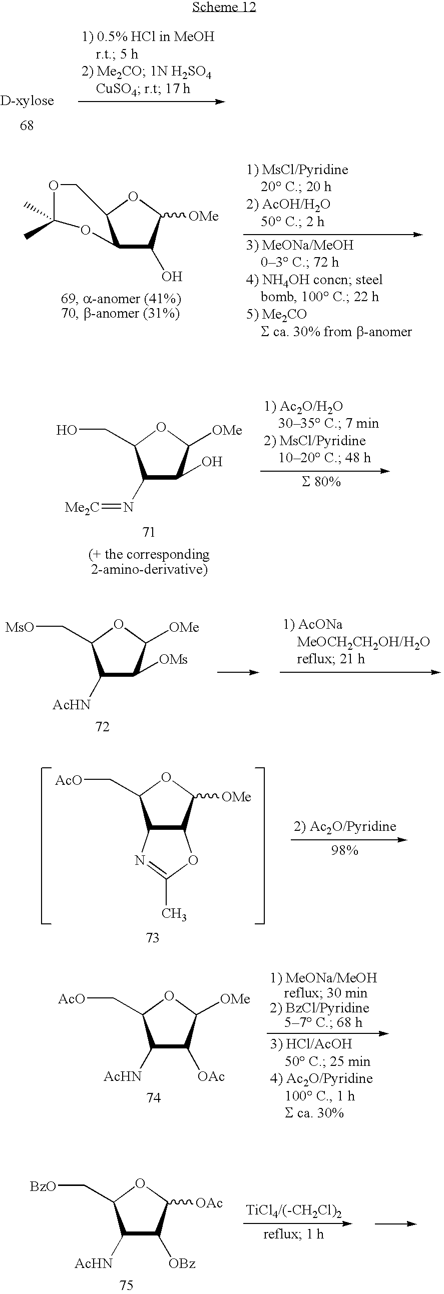

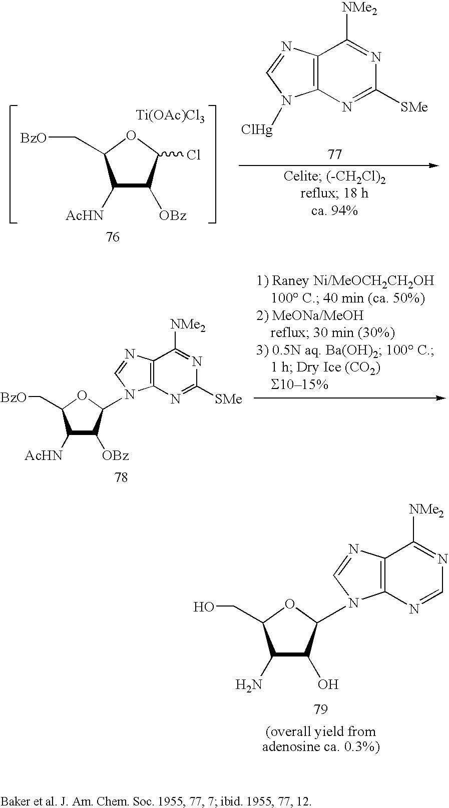

Modification of this route was published simultaneously [Baker, B. R. et al. J. Am. Chem. Soc. 1955, 77, 5905-5910] and it consists in the use of crystalline 2,5-di-O-benzoyl-3-phtalimido-3-deoxy-β-D-ribofuranosyl chloride (83) as an universal glycosylating agent. It was prepared from methyl 3-acetamido-2,5-di-O-acetyl-3-deoxy-β-D-ribofuranoside (74) that, in turn, was prepared from D-xylose as shown on Scheme 12. Compound 74 was deacetylated in two steps to afford methyl glycoside 80 that was treated with phtalic anhydride in DMF, the resulting derivative 81 was benzoylated and then underwent to acetolysis to give α-D-acetate (82). The latter was treated with ether saturated with HCl in the presence of acetyl chloride at 0° C. for 72 h and the desired 83 was precipitated from the reaction mixture (ca. 1.5% combined yield from D-xylose) (Scheme 13).

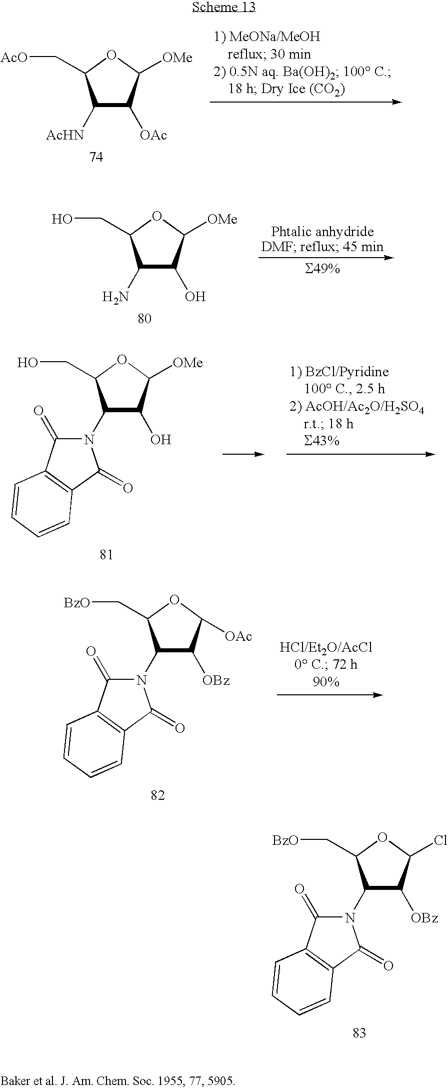

Chloride 83 had been used for the synthesis of few purine [Kissman, H. M. et al. J. Med. Chem. 1963, 6, 407-409; Goodman, L. et al. J. Med. Chem. 1963, 6, 413-423] and pyrimidine [Kissman, H. M. et al. J. Am. Chem. Soc. 1958, 80, 2575-2583] nucleosides. However, very laborious syntheses of both glycosylating agents 75 and 83 precluded them from broad application.

Later, two more efficient approaches have been suggested that use D-xylose as starting material [Azhayev, A. A et al. Coll. Czech. Chem. Comm. 1978, 43, 1520; Azhayev, A. A et al. Nucl. Acids Res. 1979, 6, 625-643; Ozols, A. M. et al., Synthesis 1980, 557-558; Okruszek, A. et al. J. Med. Chem. 1979, 22, 882-885]. D-Xylose (68) was transformed to 1,2-O-isopropylidene-α-D-xylofuranose (84) [Baker, B. R. et al. J. Am. Chem. Soc. 1955, 77, 5900-5905] that was selectively 5-O-toluylated to give compound 85. Activation of the 3-hydroxyl group by trifluoromethanesulfonylation (Tfl) [supra, Synthesis 1980,] was found to be advantageous over tosylation [supra, B.P. Nucl. Acids Res. 1979, 6]. Treatment of the triflate 86 with LiN₃ in boiling EtOH led to formation of azide 88 (51%) along with of olefin 87 (41%) resulting from 1,2-trans-elimination of triflic acid. Removal of isopropylidene group of the former followed by acetylation gave azide 89 (combined yield from D-xylose 30%) that was used as an universal glycosylating agent for the synthesis of 3-amino-3-deoxy-β-D-ribofuranosyl nucleosides of adenine (57), uracil (90) and cytosine (91) in good yields (Scheme 14).

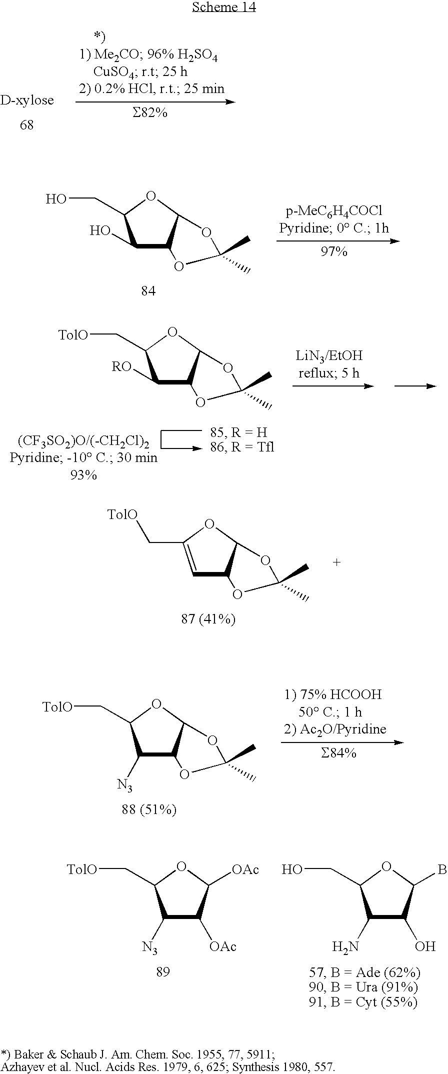

Recently, this method was employed to the synthesis of D- and L-enantiomers of 3′-azido- and 3′-amino-3′-deoxy adenosine with some modifications [Botta, O. et al. Tetrahedron 1998, 54, 13529-13546].

The other approach [supra, J. Med. Chem. 1979, 22] also used D-xylose as starting compound that was first transformed to benzoate 92 (Scheme 15) [Tong, G. L. et al. J. Org. Chem. 1967, 32, 1984-1986]. Its oxidation followed by oxymation, stereospecific reduction of oxime 94 to amine 95 with inversion of configuration at C3 [Fujiwara, A. N. et al. J. Heterocycl. Chem. 1970, 7, 891], and acylation gave benzoate 96 as a mixture of α- and β-anomers. The latter was reacted with silylated N⁶-benzoyladenine (97) in the presence of SnCl₄ to afford, after deprotection, 3′-amino-3′-deoxyadenosine (57).

### 3-Amino-2,3-dideoxy-β-D-ribofuranosyl nucleosides

Chemical modification of the natural nucleosides. First experiments in this direction have been performed in the purine nucleosides. From the studies on the episulfonium ion migrations, it was predicted by Baker and co-workers [Goodman, L. et al. J. Am. Chem. Soc. 1958, 80, 1680] that this approach can be employed for the preparation of 2′-deoxy-β-D-ribonucleosides. Shortly after, this was realized by the synthesis of 2′-deoxyadenosine and it was reasonably suggested that this method can be used for the preparation of sugar modified nucleosides [Anderson, C. D. et al J. Am. Chem. Soc. 1959, 81, 3967-3974]. Indeed, this idea was realized in the first synthesis of 3′-amino-2′,3′dideoxyadenosine (101) [Lee, W. W. et al. J. Am. Chem. Soc. 1961, 83, 1906-1911] (Scheme 16). It is noteworthy that numerous studies on the episulfonium ion migrations in a series of sugars and nucleosides preceded this work.

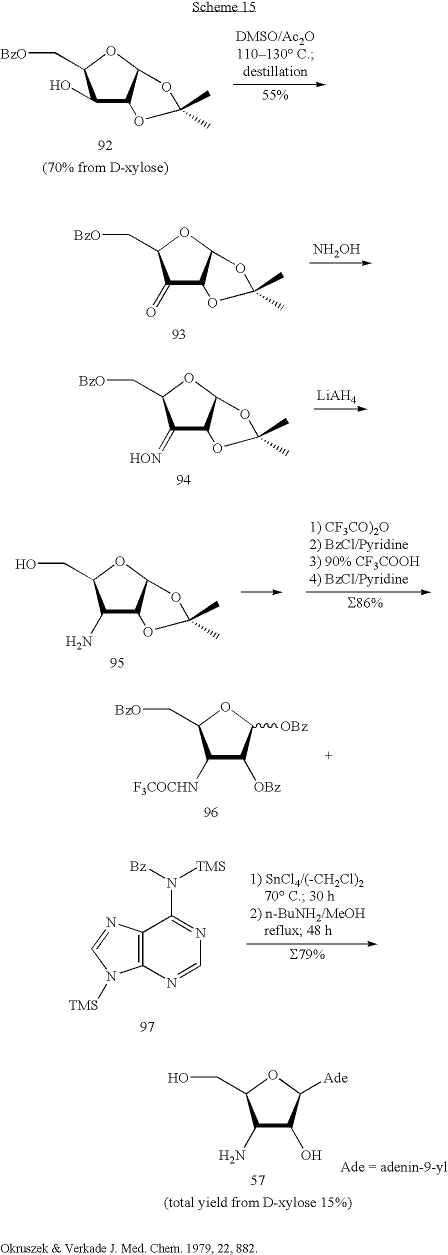

The starting point for the synthesis of 3′-amino-2′,3′-dideoxyadenosine (101) was the chloroethylthio nucleoside 98 that was obtained from 9-(2,3-anhydro-β-D-ribofuranosyl)-adenine (59) in two steps on treatment consecutively with EtSNa in EtSH (reflux under nitrogen for 19 h) and then with SOCl₂ (under nitrogen at 0° C. e 20° C. for 0.5 h) (57%; combined). The treatment of 98 with NaN₃ in refluxing aq. 2-methoxyethanol for 5 h gave a mixture of arabino 99 and xylo 100 azides (82%) that was separated into individual nucleosides by crystallization from EtOAc in 54 and 11% yield, respectively. The arabino nucleoside 99 was reduced to 3′-amino-2′,3′-dideoxyadenosine (101) (56%) under a hydrogen atmosphere in DMF in the presence of Raney nickel for 20 h. Similarly, reduction of the xylo isomer 100 gave 2′-amino-2′,3′-dideoxyadenosine (102) [supra, Am. Chem. Soc. 1961, 83] (Scheme 16).

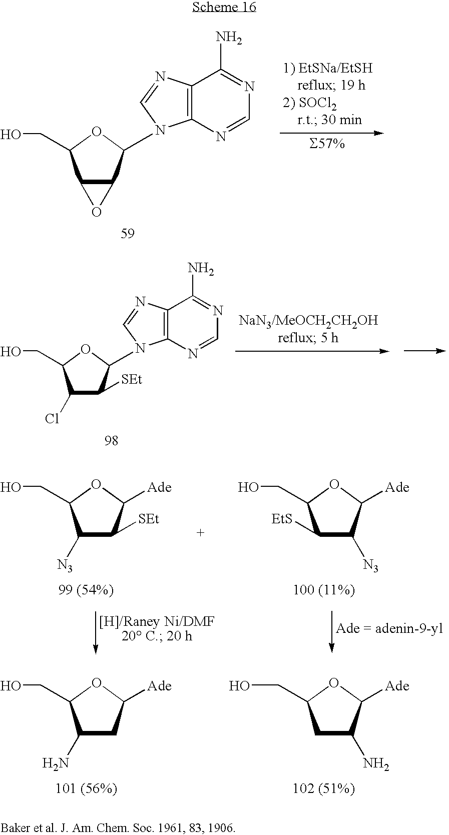

Synthesis of 3′-amino-3′-deoxythymidine (106) and 3′-azido-3′-deoxythymidine (AZT; 108) have been reported simultaneously from two laboratories [Miller, N. et al. J. Org. Chem. 1964, 29, 1772-1776; Horwitz, J. P. et al. J. Org. Chem. 1964, 29, 2076-2078]. Miller and Fox described an one-pot transformation of 3′-O-methanesulfonyl-5′-O-tritylthymidine (104) that was earlier prepared by Michelson and Todd [J. Chem. Soc. 1955, 816] from thymidine (103) to 106 through intermediate formation O²,3′-anhydro-5′-O-tritylthymidine (105) [supra, J. Org. Chem. 1964, 29] (Scheme 17).

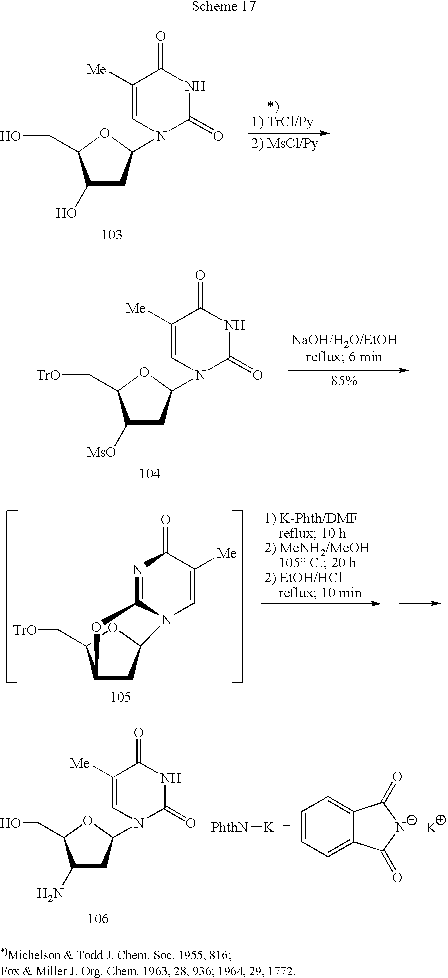

Horwitz and co-workers studied the nucleophilic substitution of 3′-O-mesyloxy group of 1-(2-deoxy-3-O-methanesulfonyl-5-O-trityl-β-D-lyxofuranosyl)thimine (107) that was earlier prepared in both laboratories by cleavage of the O²,3′-anhydro-ring of individually isolated 105 under basic conditions [supra, J. Org. Chem. 1964, 29, Fox, J. J. et al. J. Org. Chem. 1963, 28, 936-941]. The treatment of 107 with LiN₃ in DMF at 100° C. for 3 h gave, after conventional detritylation, AZT (108) (62%) (Scheme 18). The latter, on catalytic reduction, afforded 3′-amino-3′-deoxythymidine (106) (57%).

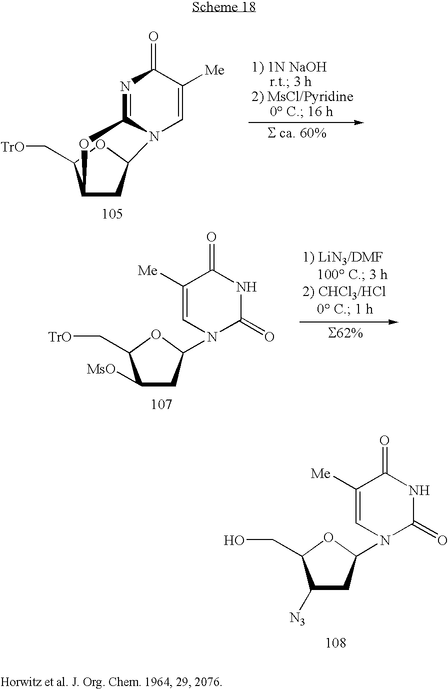

Since these first publications on the synthesis of 3′-amino-3′-deoxythymidine (106) and AZT (108), a number of papers appeared describing diverse modifications, which essentially improved and simplified the preparation of both nucleosides. Discovery of anti HIV activity of AZT and its licensing as a very important drug gave additional strong stimulus to R&D of technology for AZT preparation. Now it is produced on multi kilogram scale and is commercially available.

Coupling of purine bases with suitable amino sugars or their precursors. An easy access to AZT allowed applying it as a donor of the pentofuranose moiety in the reaction of chemical transglycosylation [Imazawa, M. et al. J. Org. Chem. 1978, 43, 3044-3048]. AZT was acetylated and then acetate 109 reacted with persilylated N⁶-octanoyladenine (110) in acetonitrile in the presence of trimethylsilyl trifluoromethanesulfonate (TMS-Tfl). Following deacylation with aq. ammonia, the mixture was chromatographed first on Dowex 1×4 (OH⁻-form) to remove all bases and thymine nucleosides, and then on silica gel. Individual N⁹-β- and N⁹-α-anomers 111 and 112 were isolated in 27 and 35% yield, respectively. As might be expected, similar condensation of acetate 109 with persilylated iv-palmitoylguanine (114) led to more complicated mixture of products. Deacylation of this mixture followed by silica gel column chromatography afforded individual 9-(3-azido-2,3-dideoxy-β-D-ribofluranosyl)guanine (115) (28%) and it's α-anomer 116 (14%), along with inseparable mixture N⁷-α- and N⁷-β-azides 117 and 118 (13%).

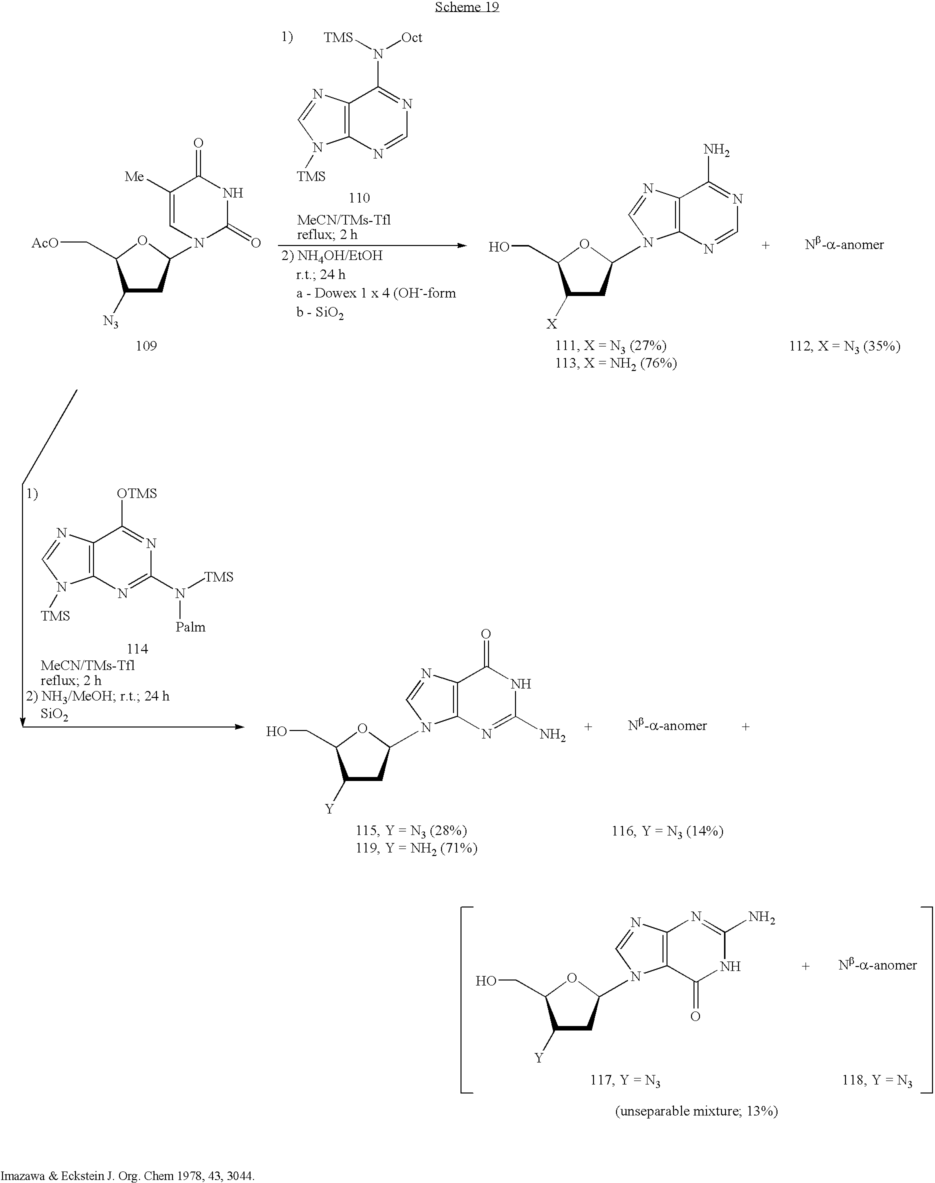

Reduction of azides 111 and 115 to the respective amines 113 (76%) and 119 (71%) was accomplished by the use of triphenylphosphine in dioxane in the presence of water (50° C.; 24 h) in high yields [supra J. Org. Chem. 1978, 43].

The first convergent synthesis of 3-azido-2,3-dideoxy-D-ribofuranosyl nucleosides of natural heterocyclic bases was realized employing methyl 3-azido-2,3-dideoxy-5-O-toluoyl-D-ribofuranoside (124) as an universal glycosylating agent [Dyatkina, N. B. et al. Synthesis 1984, 961-963; Dyatkina, N. B. et al. Bioorg. Chem. (Moscow) 1986, 12, 1048-1053]. It was prepared from readily available 1,2-O-isopropylidene-α-D-xylofuranose (84). Exhaustive benzylation of 84 followed by methanolysis of isopropylidene group gave methyl xyloside 120. The latter was transformed into dithiocarbonate 121 by the reaction first with CS₂ and then with methyl iodide, which was treated with n-Bu₃SnH (Barton reductive deoxygenation) to give 122. Its debenzylation followed by selective 5-O-toluylation and then 3-O-acylation with triflic anhydride led to triflate 123. Nucleophilic substitution of trifliloxy group under very mild conditions gave the desired glycosylating agent 124. Glycosylation of silylated bases in the presence of Lewis acids led to formation of nucleosides in moderate yields (Scheme 20). It is interesting to note that no formation of α-anomers and N⁷-isomers of adenine and guanine nucleosides 111 and 115 was observed.

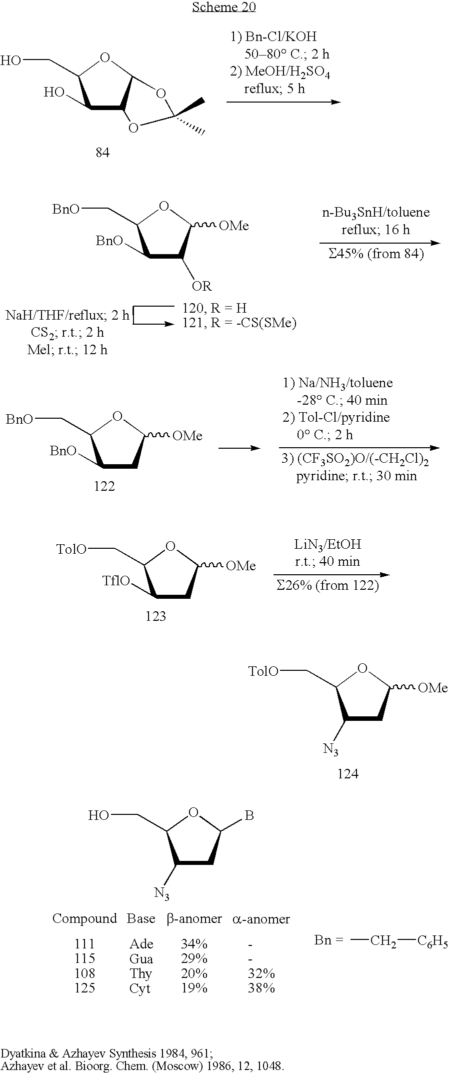

Miscellaneous. Suhadolnik and co-workers have isolated four nucleoside antibiotics, viz., 9-(β-D-arabinofuranosyl)adenine (ara-A), 2′-chloro-2′-deoxycoformycin, 2′-amino-2′-deoxyadenosine and nucleocidin from the culture filtrate of Actinomadura and Streptomyces clavus [Suhadolnik, R. J. et al. Nucleosides Nucleotides 1988, 8, 983-986] and established the biosynthesis pathways leading to these antibiotics [Suhadolnik, R. J. et al. Arch. Biochem. Biophys. 1989, 270, 374-382]. There were no reports on preparative synthesis of these antibiotics.

Gerber and Lechevalier reported on the isolation of 3′-amino-3′-deoxyadenosine from a cultivation of Helminthosporum sp. 215 in a sucrose-containing medium, but yield was low and the purification of the nucleoside was very laborious [Gerber, N. N. et al. J. Org. Chem. 1962, 27, 1731-1732].

### BRIEF SUMMARY OF THE INVENTION

The present invention relates to a method of preparing aminodeoxy purine N⁹-β-D-nucleosides containing 3-amino-3-deoxy-β-D-ribofuranose, 3-amino-2,3-dideoxy-β-D-ribofuranose, and 2-amino-2-deoxy-β-D-ribofuranose as sugar moieties of general formulas I, II and III, respectively.

More specifically, the present invention is directed to a biocatalytic process for preparing purine aminodeoxy N⁹-β-D-nucleosides comprising reacting at least one purine base with at least one donor of a carbohydrate moiety in the presence of a biocatalyst wherein the purine base undergoes regio- and stereo-selective transglycosylation. The purine aminodeoxy N⁹-β-D-nucleoside preferably is selected from the group consisting of formulas I, II and III, 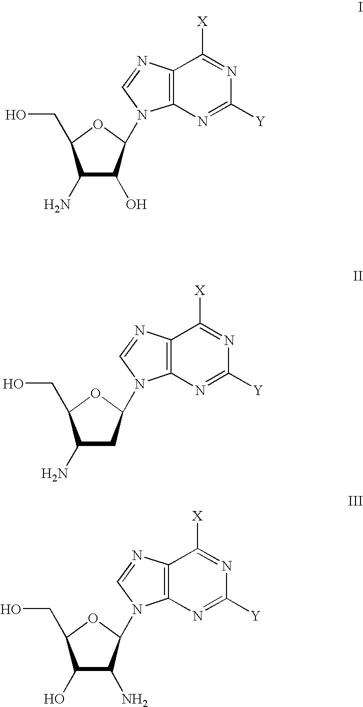
wherein X is selected from hydrogen, an amino group, chlorine, a mercapto group, or hydroxyl and Y is selected from hydrogen or an amino group.

In one embodiment, the at least one donor of a carbohydrate moiety is at least one selected from the group consisting of 3′-amino-3′-deoxyuridine, 3′-amino-3′-deoxythymidine and 2′-amino-2′-deoxyuridine.

In one embodiment the biocatalyst may be selected from bacterial cells or glutaraldehyde (GA) treated bacterial cells including Escherichia coli and glutaraldehyde (GA) treated cells of Escherichia coli. Alternatively, the biocatalyst may be selected from pure enzymes isolated from the bacterial cells such as thymidine phosphorylase, uridine phosphorylase, or mixtures thereof, combined with purine nucleoside phosphorylase.

In another embodiment, the reaction occurs in the presence of a suitable buffer such as potassium phosphate or sodium phosphate.

### DETAILED DESCRIPTION OF THE INVENTION

The present invention is directed to a method of preparing aminodeoxy purine N⁹-β-D-nucleosides containing 3-amino-3-deoxy-β-D-ribofuranose, 3-amino-2,3-dideoxy-β-D-ribofuranose, and 2-amino-2-deoxy-β-D-ribofuranose as sugar moieties of general formulas I, II and III, respectively.

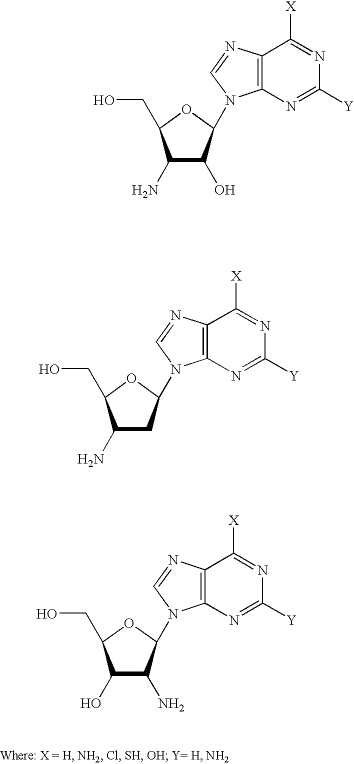

Specifically, the present invention relates to a method of producing aminodeoxy purine N⁹-β-D-nucleosides by stereo- and regio-specific biocatalytic transglycosylation of purine bases using at least one donor of a carbohydrate moiety, in particular, 3′-amino-3′-deoxyuridine, 3′-amino-3′-deoxythymidine or 2′-amino-2′-deoxyuridine, in the presence of a biocatalyst such as cells of Escherichia coli, or the glutaraldehyde (GA) treated cells of Escherichia coli or pure enzymes (uridine phosphorylase, thymidine phosphorylase, or mixtures thereof and combined with purine nucleoside phosphorylase).

Chemical methods for preparing 3′-amino-3′-deoxyuridine, 3′-amino-3′-deoxythymidine and 2′-amino-2′-deoxyuridine are known and rather efficient. On the contrary, known syntheses methods for preparing relevant purine nucleosides are multistage, laborious, and low-yielding.

It is known that the combination of chemical and enzyme-catalyzed procedures can be an efficient route for the synthesis of purine nucleosides modified in pentofuranose residue and/or heterocyclic base employing available pyrimidine nucleosides as donors of carbohydrate moiety and purine bases as acceptors in an enzymatic reaction (see, e.g., [Barai, V. N. et al. Helv. Chim. Acta 2002, 85, 1893-1900]. It was shown in Zaitseva, G. V et al. Nucleosides & Nucleotides 1994, 13, 819-834 that 3′-amino-3′-deoxythymidine (106) can be used as a donor of the carbohydrate moiety in the reaction of enzymatic transglycosylation of adenine (126) and guanine (127) to afford 3′-amino-2′,3′-deoxyadensine (113) and 3′-amino-2′,3′-deoxyguanosine (119), respectively.

The ratio of substrates, 106 and 126, in the synthesis of 113 was 1:3, reaction was conducted at 50° C. for 24 h in the presence of glutaraldehyde-treated cells of E. coli BM-11 to afford 113 in 46% yield, thereby 23% of starting 106 was recovered. In the case of 119, the 1.0:1.09 ratio of the substrates was employed, reaction was conducted at 50° C. for 28 h in the presence of the whole E. coli cells BMT-38 to give 119 in 20% yield and 59% of 106 was recovered (Scheme 21). Both strains of E. coli (BM-11) and (BM-38) were employed as biocatalysts. The inventors are unaware of any other reports in the literature on the enzymatic synthesis of purine aminodeoxy nucleosides.

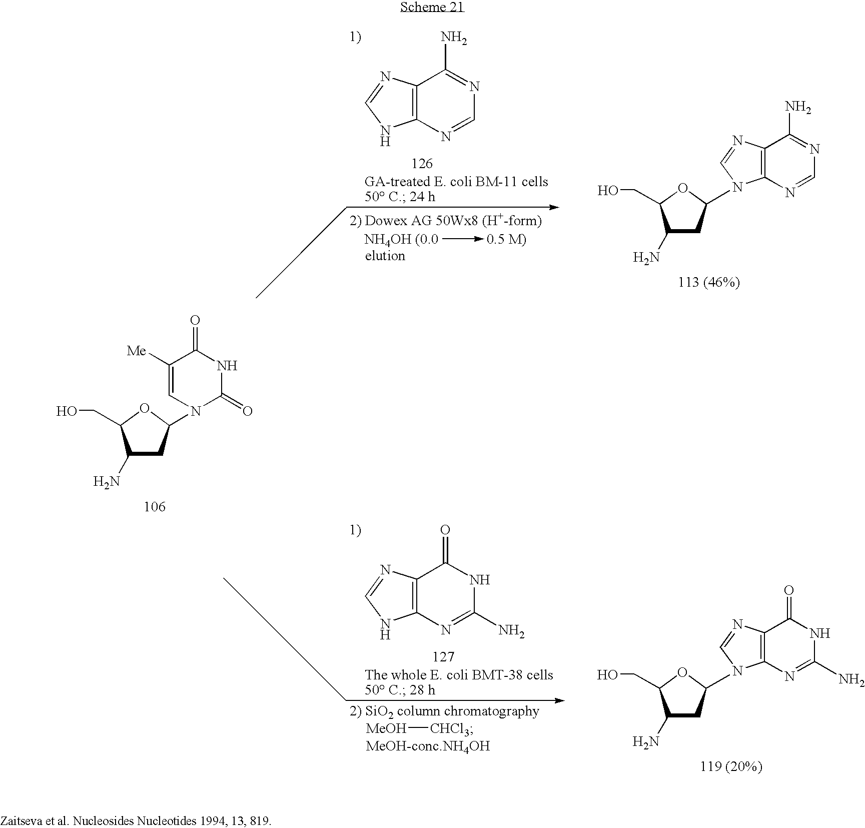

The present invention demonstrates that purine aminodeoxy nucleosides can be uniformly prepared by the stereo- and regeospecific biocatalytic transglycosylation of purine bases employing the respective 3′-amino-3′-deoxyuridine, 3′-amino-3′-deoxythymidine and 2′-amino-2′-deoxyuridine as donors of the carbohydrate moiety. The biocatalysts discovered to be useful with the process include whole cells of Escherichia coli, glutaraldehyde (GA) treated cells of Escherichia coli, the corresponding pure enzymes—thymidine phosphorylase, uridine phosphorylase, or mixtures thereof, together with purine nucleoside phosphorylase, isolated from the bacterial cells. Optionally, enzymes may be immobilized on the solid phase.

Experiments directed towards the selection of the E. coli cells resulted in the production of two different strains—1K/1T and 2K/2T, whole cells of which display high enzymatic activities involved in the transglycosylation process under consideration, viz., thymidine phosphorylase (TPase) and purine nucleoside phosphorylase (PNPase) in the case of 1K/1T strain and uridine phosphorylase (UPase) and purine nucleoside phosphorylase (PNPase) in the case of 2K/2T strain. The activity of the whole cells of 1K/1T strain as such, or treated with glutaraldehyde, is illustrated by the transglycosylation of adenine (126; a 106:126 ratio is 2:1, mol) and 2,6-diaminopurine (DAP; 128; a 106:128 ratio is 2:1, mol) in the presence of phosphate buffer (10-50 mM, in particular 20-30 mM, potassium, sodium phosphate, pH 6-9) at elevated temperature (40-55° C.) for 16-40 h. These reactions afforded 3′-amino-2′,3′-dideoxyadenosine (113) and 9-(3-amino-2,3-dideoxy-β-D-ribofuranosyl)-2,6-diaminopurine (DAP nucleoside; 129) (Scheme 22). The latter is well known mimic of adenine in the formation of base pair with thymine and efficiency of the DAP-T base pair is much higher vs. the natural A-T pair and comparable with that of the G-C pair. It can be, therefore, used instead of adenosine nucleoside for the preparation of oligonucleotides with enhanced thermodynamic properties, viz., higher thermal stability. Moreover, DAP nucleoside 129 is a versatile intermediate for the preparation of diverse purine nucleoside by means of modification of the heterocyclic base employing well known reactions. In this context, deamination under the action of readily available adenosine deaminase (ADase) affords the corresponding guanine nucleoside 119.

Similar biocatalytic regio- and stereoselective syntheses of aminodeoxy purine N⁹-β-D-nucleosides containing 3-amino-3-deoxy-β-D-ribofuranose (type I) and 2-amino-2-deoxy-β-D-ribofuranose (type III) as sugar moieties have been performed, using the 1:1 mixture of whole cells of 1K/1T strain and whole cells of 2K/2T as such, or both treated with glutaraldehyde, in the presence of phosphate buffer (10-50 mM, particularly 20-30 mM, potassium or sodium phosphate, pH 6-9) at 45-60° C. for 45-200 h.

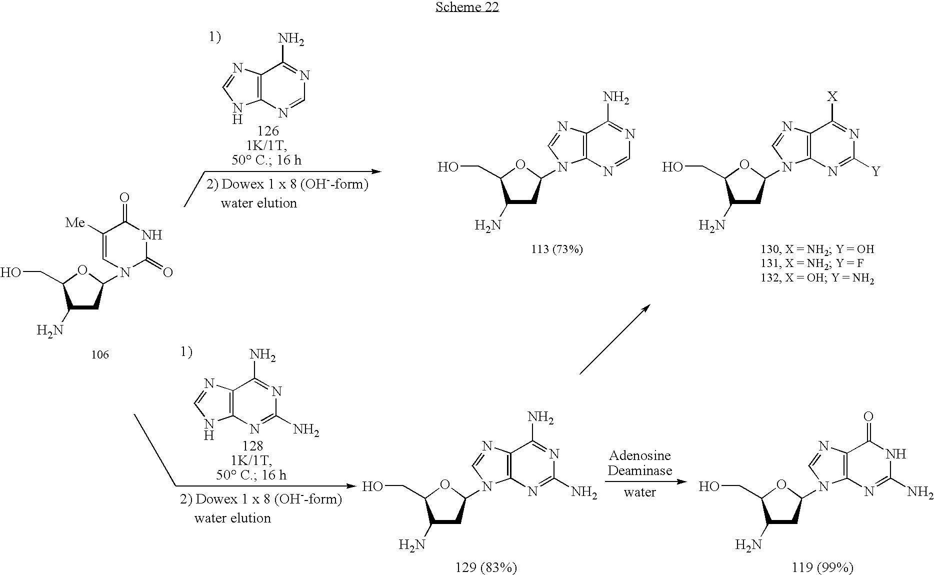

In toto, biocatalytic synthesis of 9-(3-amino-2,3-deoxy-β-D-ribofuranosyl)purines 113 and 119 are an essential and unexpected improvement of the previously published method [supra, Nucleosides & Nucleotides 1994, 13]. The novel biocatalytic synthesis of DAP-nucleoside 129 represents a new strategy in preparing key intermediates for numerous enzymatic and chemical modifications. Aminodeoxy purine N⁹-β-D-nucleosides containing 3-amino-3-deoxy-β-D-ribofuranose (type I) and 2-amino-2-deoxy-β-D-ribofuranose (type III) as sugar moieties have been biocatalytically regio- and stereoselective synthesized.

A purine base is combined with a donor of a carbohydrate moiety in a mole ratio of 0.1-1.5:1, preferably about 0.5:1. An effective amount of a biocatalyst is added to the mixture to catalyze regio- and stereo-selective transglycosylation. A suitable amount of a buffer is added to achieve a pH level of 6 to 9, preferably 6.5 to 8.5. The mixture is incubated at a temperature of 40 to 70° C. for 16-200 h until a constant amount of corresponding desired aminodeoxy purine nucleoside is achieved.

The biocatalyst is separated by any suitable means such as centrifugation. The product is then isolated, for example, employing chromatography on ion exchange resins.

The present invention provides uniform preparation of all types of purine aminodeoxy nucleosides allowing the possibility to synthesize the purine nucleosides on preparative level, from multigrams to multikilograms, making the purine nucleosides available for any practical applications with reasonable prices.

In addition, the synthesis of the purine nucleosides allows a more efficient process of producing oligonucleotides. Oligonucleotides bearing different reporting groups and other functional entities have become a commonplace tool in many diagnostic and therapeutic applications. Generally, synthesis of functionalized oligonucleotides is implemented by direct introduction of a protected functionality via phosphoramidite solid phase chemistry. This approach requires the preparation of the corresponding monomer for every new functionality to be incorporated.

The 9-(2-amino-2-deoxy-β-D-ribofuranosyl)purines may be used as starting compounds for preparing such monomers since the purines have very active 2′-amino function for further modifications (see, e.g., [Benzeler, F. et al. Nucleosides & Nucleotides 1992, 11, 1333-1351; Eckstein, F. Biochimie 2002, 84, 841-848. Aurup, H. et al. Nucl. Acids Res. 1994, 22, 20-24; Verma, S. et al. Annu. Rev. Biochem. 1998, 67, 99-134; Menger, M. et al. Biochemistry 2000, 39, 4500-4507; Polushin, N. et al. Nucleosides Nucleotides Nucleic Acids 2001, 20, 507-514 and Polushin, N. et al. Nucleosides Nucleotides Nucleic Acids 2001, 20, 973-976].

In addition, the synthesis of structural purine building blocks for the oligonucleotide N3′→P5′ phosphoramidates typically starts from the natural 2′-deoxyadenosine and 2′-deoxyguanosine and requires 10 stages leading to key precursor for the preparation of phosphoramidite building blocks in ca. 20% yield [Nelson, J. S. et al. J. Org. Chem. 1997, 62, 7278-7287]. Whereas, starting from 9-(3-amino-2,3-deoxy-β-D-ribofuranosyl)purines 113 and 119, the same phosphoramidite building blocks may be prepared in 5-6 steps with an expected yield of 30-40%. Thus, oligonucleotide N3′→P5′ phosphoramidates with the 9-(3-amino-2,3-deoxy-β-D-ribofuranosyl)purines saves process steps and provides higher yield.

The present invention may be used for diverse areas of biological and medicinal applications.

The following Examples illustrate the present invention but should not be considered in any way limiting of the invention.

### EXAMPLE 1

### Synthesis 3′-amino-3′-deoxythymidine (3′NH₂-dThd)

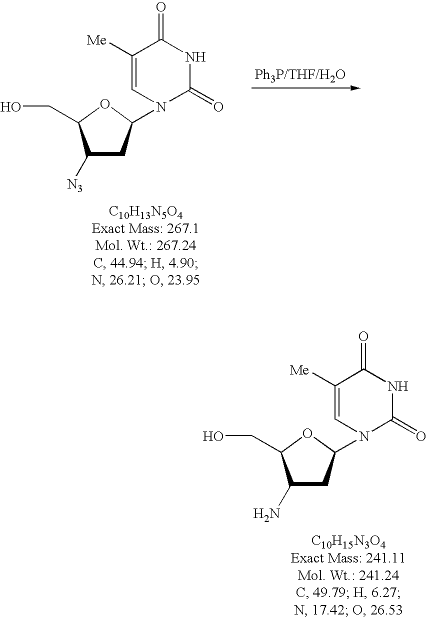

To a stirred solution of AZT (100 g, 0.374 mol) in a mixture of THF (850 mL) and water (20 mL) at 40-50° C. was added dropwise during 3 h a solution of Ph₃P (106 g, 0.404 mol; molar ratio is 1.0:1.08) in THF (300 mL) and the reaction mixture was stirred for 24 h. Thin crystalline product formed was filtered off, washed with THF (250 mL) and dried on air for 48 h to give 79 g (87.5%) of TLC pure 3′NH₂-dThd.

Combined mother liquor and washing were evaporated to dryness to give 128 g of a mixture that was partitioned between water (300 mL) and CH₂Cl₂ (700 mL), water phase was evaporated to give 3′NH₂-dThd as a main component along with Ph₃PO (14 g). It was crystallized from a mixture of EtOH (140 mL), MeOH (120 mL) and water (8 mL) under reflux, followed by cooling at +4° C. during a week gave, after filtration of thin crystalline product and washing with abs. ETOH (2×20 mL), CH₂Cl₂ (2×20 mL), and ether (2×20 mL), 9.5 g of TLC pure 3′NH₂-dThd. Combined yield 98%.

### EXAMPLE 2

### Synthesis 3′-amino-2′,3′-dideoxyadenosine (3′NH₂-ddAdo)

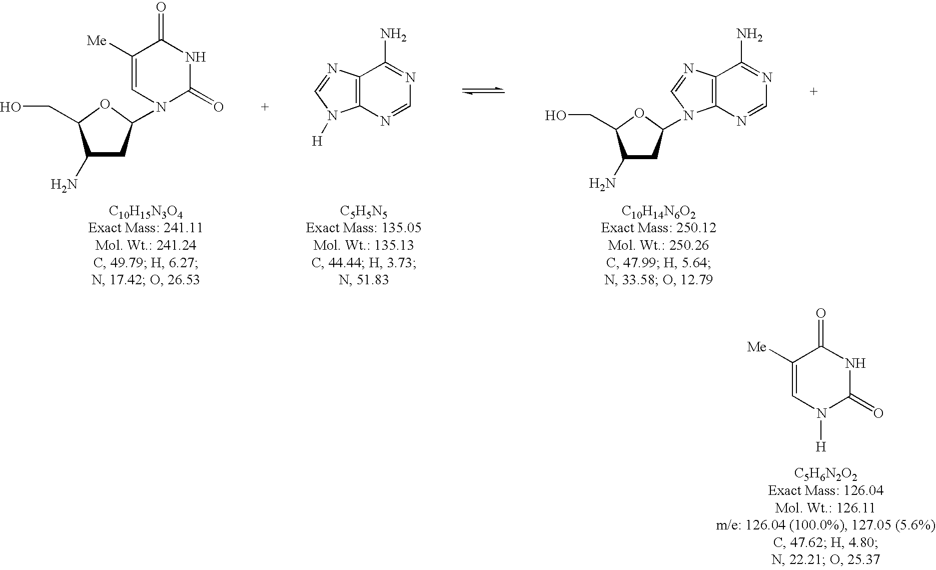

A reaction mixture (100 mL) containing 3′NH₂-dThd (2.42 g, 0.01 mol), adenine (0.68 g, 0.005 mol), potassium phosphate buffer (20 mM, pH 6.0) and biocatalyst (E. coli cells 1K/1T or glutaraldehyde (GA) treated cells E. coli cells 1K/1T; 3.0 g wet weight; 0.6 g dry weight) or pure thymidine phosphorylase (1250 IU) with purine nucleoside phosphorylase (950 IU) was incubated at 50° C. with gentle stirring for 16-24 h, cooled down till room temperature and placed into a refrigerator at 4° C. for 24 h. The biocatalyst was separated by centrifugation (5,000×g; 10 min), treated with water (20 mL) at room temperature and again centrifuged under the same conditions. The supernatants were combined and applied onto a column (3.0×5.7 cm; 40 mL) with Dowex 1×8 (200-400 mesh, OH⁻-form). The column was eluted with water; the product containing fractions were combined (ca. 150 mL), evaporated at 50° C. to dryness and co-evaporated with abs. EtOH to give 0.87-0.95 g (69-75%) of 3′NH₂-ddAdo that was individual according to the TLC, HPLC, UV and ¹H and ¹³C NMR data.

### EXAMPLE 3

### Synthesis 2-amino-(3-amino-2,3-dideoxy-β-D-ribofuranosyl)adenine (3′NH₂-ddDAP) and 3′-amino-2′,3′-dideoxyguanosine (3′NH₂-ddGuo)

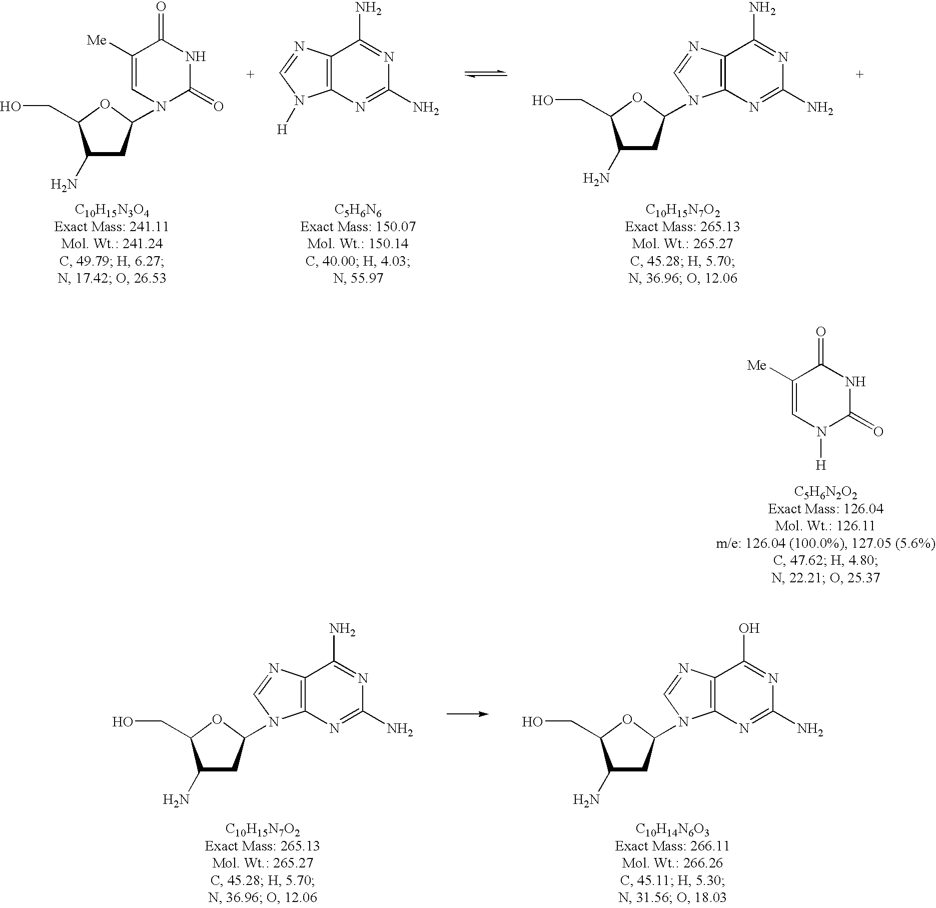

A reaction mixture (100 mL) containing 3′NH₂-dThd (2.42 g, 0.01 mol), 2,6-diaminopurine (0.75 g, 0.005 mol), potassium phosphate buffer (20 mM, pH 6.0) and biocatalyst (E. coli cells 1K/1T or glutaraldehyde (GA) treated cells E. coli cells 1K/1T; 3.0 g wet weight; 0.6 g dry weight) or pure thymidine phosphorylase (1250 IU) with purine nucleoside phosphorylase (950 IU), was incubated at 50° C. with gentle stirring for 16-24 h, cooled down till room temperature and placed into a refrigerator at 4° C. for 24 h. The biocatalyst was separated by centrifugation (5,000×g; 10 min), treated with water (20 mL) at room temperature and again centrifuged under the same conditions. The supernatants were combined and applied onto a column (3.0×5.7 cm; 40 mL) with Dowex 1×8 (200-400 mesh; OH⁻-form). The column was eluted with water; the product containing fractions were combined (ca. 150 mL), evaporated at 50° C. to dryness and co-evaporated with abs. EtOH to give 1.04-1.15 g (78-87%) of 3′NH₂-ddDAP that was individual according to the TLC, HPLC, UV and ¹H and ¹³C NMR data.

The 3′NH₂-ddDAP (2.65 g, 0.1 mol) was dissolved under gentle heating (<50° C.) in water (ca. 60 mL) in glass, cooled to r.t., adenosine deaminase (15-20 μL; from calf intestine, 10 mg in 2 mL, Boehringer Mannheim, Germany) was added and the reaction mixture was incubated under stirring till complete disappearance of the starting nucleoside [TLC monitoring (silica gel 60 F₂₅₄; EtOAc—BuOH—H₂O, 10:5:1, vol): R_{f} values of 3′NH₂-ddDAP and 3′NH₂-ddGuo are 0.32 and 0.18, respectively] (24-48 h) and formation of white precipitate of 3′NH₂-ddGuo. Reaction mixture was evaporated at 50° C. to 50 mL, stored in refrigerator at 4° C. for 24-48 h, precipitated product was filtered off, washed with water (2×3-4 mL), EtOH (2×5 mL) a dried for 2 h at 80° C. to give 2.46 g (88%) of 3′NH₂-ddGuo as monohydrate that was individual according to the TLC, HPLC, UV and ¹H and ¹³C NMR data.

### EXAMPLE 4

### Synthesis 3′-amino-3′-deoxyadenosine (3′NH₂-dAdo)

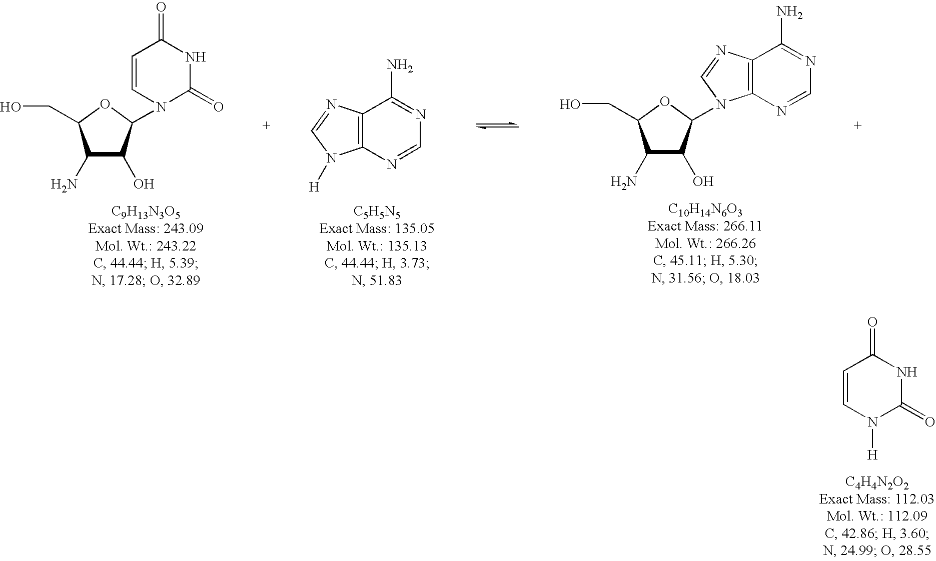

A reaction mixture (100 mL) containing 3′-amino-3′-deoxyuridine (2.44 g, 0.01 mol), adenine (0.68 g, 0.005 mol), potassium phosphate buffer (30 mM, pH 7.0) and biocatalyst (a mixture of E. coli cells 1K/1T or glutaraldehyde (GA) treated cells E. coli cells 1K/1T; 3.0 g wet weight; 0.6 g dry weight and E. coli cells 2K/2T or glutaraldehyde (GA) treated cells E. coli cells 2K/2T; 3.0 g wet weight; 0.6 g dry weight) or pure uridine phosphorylase (2500 IU) with purine nucleoside phosphorylase (2000 IU) was incubated at 60° C. with gentle stirring for 125-150 h, cooled down till room temperature and placed into a refrigerator at 4° C. for 24 h. The precipitate containing biocatalyst and heterocyclic bases was separated by centrifugation (5,000×g; 10 min), treated with water (20 mL) at room temperature and again centrifuged under the same conditions. The supernatants were combined and applied onto a column (3.0×7.1 cm; 50 mL) with Dowex 50×8 (200-400 mesh; NH₄⁺-form). The column was eluted with 0.2 M NH₄OH; the product containing fractions were combined (ca. 150 mL), evaporated at 50° C. to dryness and co-evaporated with abs. EtOH. The powder was suspended in 5 ml abs. EtOH (20 h, r.t.), filtered and dried (70° C., 5 h) to give 0.77-0.86 g (58-65%) of 3′NH₂-3′dAdo that was individual according to the TLC, HPLC, UV and ¹H and ¹³C NMR data.

### EXAMPLE 5

### Synthesis 2-amino-(3-amino-3-deoxy-β-D-ribofuranosyl)adenine (3′NH₂-ddDAP) and 3′-amino-3′-deoxyguanosine (3′NH₂-ddGuo)

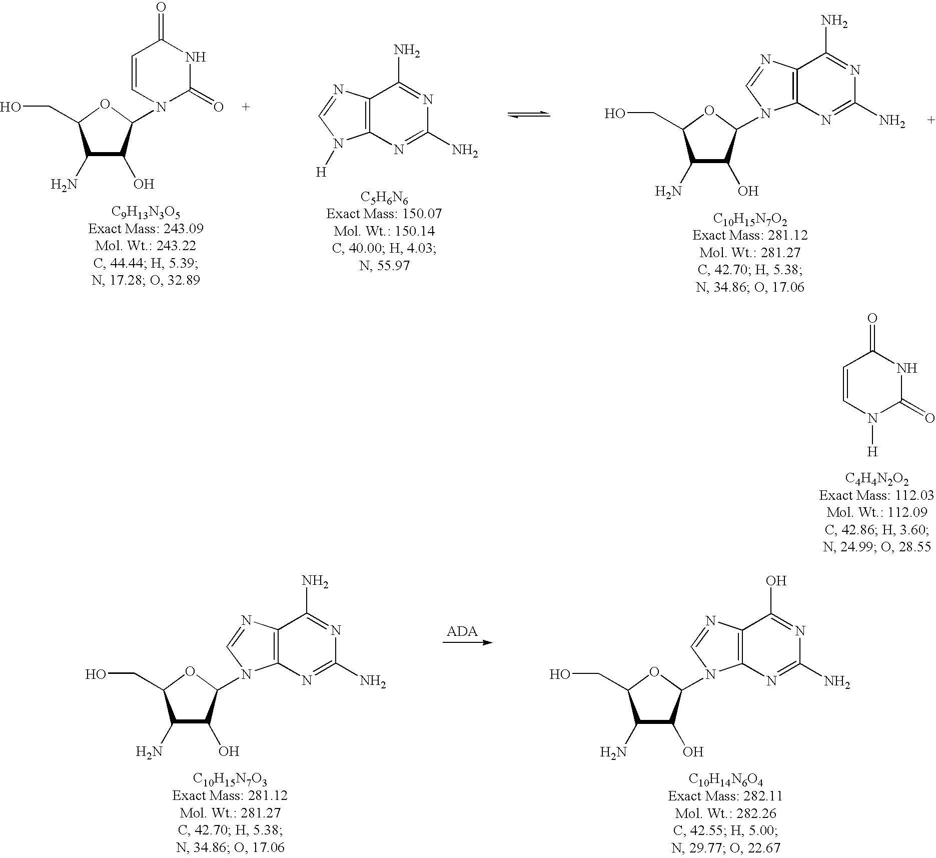

A reaction mixture (100 mL) containing 3′-amino-3′-deoxyuridine (2.44 g, 0.01 mol), 2,6-diaminopurine (0.75 g, 0.005 mol), potassium phosphate buffer (30 mM, pH 7.0) and biocatalyst (a mixture of E. coli cells 1K/1T or glutaraldehyde (GA) treated cells E. coli cells 1K/1T; 3.0 g wet weight; 0.6 g dry weight and E. coli cells 2K/2T or glutaraldehyde (GA) treated cells E. coli cells 2K/2T; 3.0 g wet weight; 0.6 g dry weight) or pure uridine phosphorylase (2500 IU) with purine nucleoside phosphorylase (2000 IU) was incubated at 60° C. with gentle stirring for 125-150 h, cooled down till room temperature and placed into a refrigerator at 4° C. for 24 h. The precipitate containing biocatalyst and heterocyclic bases was separated by centrifugation (5,000×g; 10 min), treated with water (20 mL) at room temperature and again centrifuged under the same conditions. The supernatants were combined and put onto a column (3.0×7.1 cm; 50 mL) with Dowex 50×8 (200-400 mesh; NH₄⁺-form). The column was eluted with 0.2 M NH₄OH; the product containing fractions were combined (ca. 150 mL), evaporated at 50° C. to dryness and co-evaporated with abs. EtOH. The powder was suspended in 5 ml abs. EtOH (20 h, r.t.), filtered and dried (70° C., 5 h) to give 0.87-0.95 g (62-68%) of 3′NH₂-dDAP that was individual according to the TLC, HPLC, UV and ¹H and ¹³C NMR data.

Deamination of 3′NH₂-dDAP under the action of ADA as it is described in Example 3 gave 3′NH₂-dGuo as amorphous powder in 95% yield.

### EXAMPLE 6

### Synthesis 2′-amino-2′-deoxyadenosine (2′NH₂-dAdo)

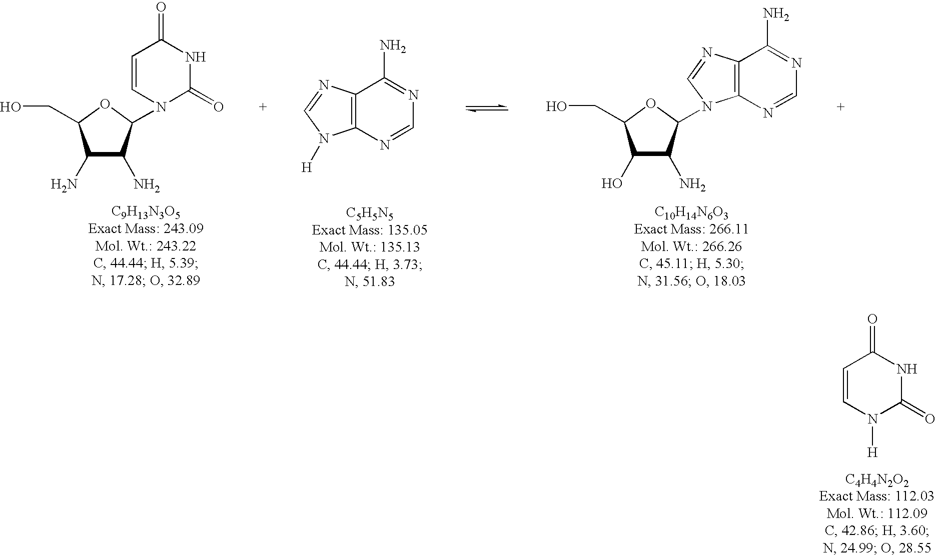

A reaction mixture (100 mL) containing 2′-amino-2′-deoxyuridine (2.44 g, 0.01 mol), adenine (0.68 g, 0.005 mol), potassium phosphate buffer (30 mM, pH 7.0) and biocatalyst (a mixture of E. coli cells 1K/1T or glutaraldehyde (GA) treated cells E. coli cells 1K/1T; 2.0 g wet weight; 0.4 g dry weight and E. coli cells 2K/2T or glutaraldehyde (GA) treated cells E. coli cells 2K/2T; 2.0 g wet weight; 0.4 g dry weight) or pure uridine phosphorylase (2000 IU) with purine nucleoside phosphorylase (1600 IU) was incubated at 60° C. with gentle stirring for 40-50 h, cooled down till room temperature and placed into a refrigerator at 4° C. for 24 h. The precipitate containing biocatalyst and heterocyclic bases was separated by centrifugation (5,000×g; 10 min), treated with water (20 mL) at room temperature. and again centrifuged under the same conditions. The supernatants were combined and put onto a column (3.0×7.1 cm; 50 mL) with Dowex 50×8 (200-400 mesh; NH₄⁺-form). The column was eluted with 0.2 M NH₄OH; the product containing fractions were combined (ca. 150 mL), evaporated at 50° C. to dryness and co-evaporated with abs. EtOH. The powder was suspended in 5 ml abs. EtOH (20 h, r.t.), filtered and dried (70° C., 5 h) to give 0.95-1.04 g (71-78%) of 2′NH₂-2′dAdo that was individual according to the TLC, HPLC, UV and ¹H and ¹³C NMR data.

### EXAMPLE 7

### Synthesis 2-amino-(2-amino-2-deoxy-β-D-ribofuranosyl)adenine (2′NH₂-ddDAP) and 2′-amino-2′-deoxyguanosine (3′NH₂-ddGuo)

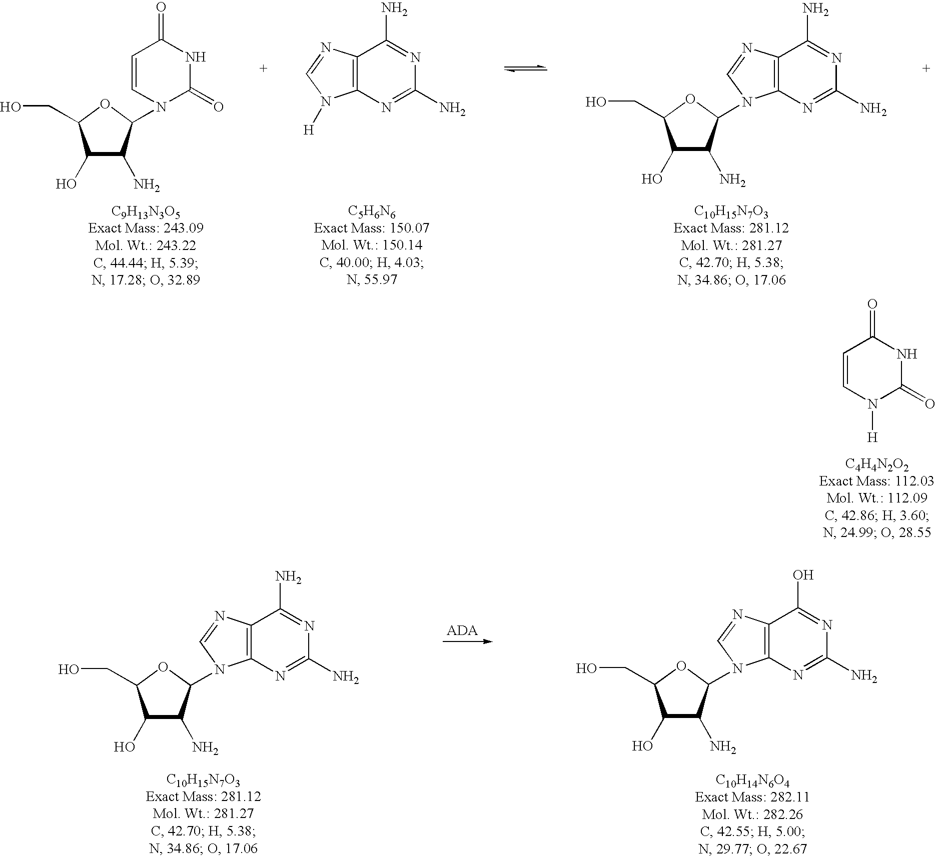

A reaction mixture (100 mL) containing 2′-amino-2′-deoxyuridine (2.44 g, 0.01 mol), 2,6-diaminopurine (0.75 g, 0.005 mol), potassium phosphate buffer (30 mM, pH 7.0) and biocatalyst (a mixture of E. coli cells 1K/1T or glutaraldehyde (GA) treated cells E. coli cells 1K/1T; 2.0 g wet weight; 0.4 g dry weight and E. coli cells 2K/2T or glutaraldehyde (GA) treated cells E. coli cells 2K/2T; 2.0 g wet weight; 0.4 g dry weight) or pure uridine phosphorylase (2000 IU) with purine nucleoside phosphorylase (1600 IU) was incubated at 60° C. with gentle stirring for 40-50 h, cooled down till room temperature and placed into a refrigerator at 4° C. for 24 h. The precipitate containing biocatalyst and heterocyclic bases was separated by centrifugation (5,000×g; 10 min), treated with water (20 mL) at room temperature and again centrifuged under the same conditions. The supernatants were combined and put onto a column (3.0×7.1 cm; 50 mL) with Dowex 50×8 (200-400 mesh; NH₄⁺-form). The column was eluted with 0.2 M NH₄OH; the product containing fractions were combined (ca. 150 mL), evaporated at 50° C. to dryness and co-evaporated with abs. EtOH. The powder was suspended in 5 ml abs. EtOH (20 h, r.t.), filtered and dried (70° C., 5 h) to give 0.93-1.0 g (66-71%) of 2′NH₂-dDAP that was individual according to the TLC, HPLC, UV and ¹H and ¹³C NMR data.

Deamination of 2′NH₂-dDAP under the action of ADA as it is described in Example 3 gave 2′NH₂-dGuo as amorphous powder in 98% yield.

We claim:

1. A biocatalytic process for preparing purine aminodeoxy N⁹-β-D-nucleosides comprising reacting at least one purine base with at least one donor of a carbohydrate moiety in the presence of a biocatalyst wherein the purine base undergoes regio- and stereo-selective transglycosylation.

2. The process of claim 1 wherein the purine aminodeoxy N⁹-β-D-nucleoside is selected from the group consisting of formulas I, II and III, 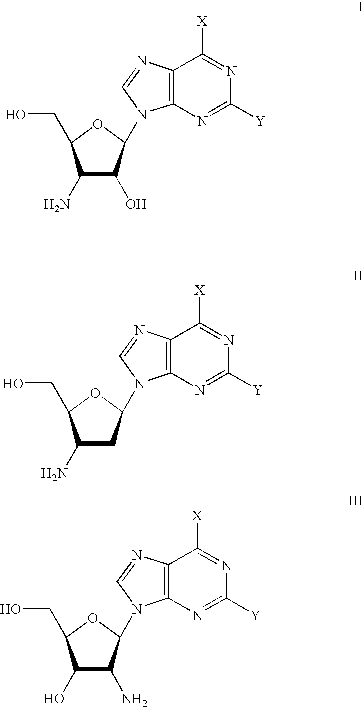
wherein X is selected from hydrogen, an amino group, chlorine, a mercapto group, or hydroxyl and Y is selected from hydrogen or an amino group.

3. The process of claim 2 wherein the purine aminodeoxy N⁹-β-D-nucleoside is selected from formula (I).

4. The process of claim 2 wherein the purine aminodeoxy N⁹-β-D-nucleoside is selected from formula (II).

5. The process of claim 2 wherein the purine aminodeoxy N⁹-β-D-nucleoside is selected from formula (III).

6. The process of claim 1 wherein the at least one donor of a carbohydrate moiety is at least one selected from the group consisting of 3′-amino-3′-deoxyuridine, 3′-amino-3′-deoxythymidine and 2′-amino-2′-deoxyuridine.

7. The process of claim 1 wherein the biocatalyst comprises bacterial cells or glutaraldehyde (GA) treated bacterial cells.

8. The process of claim 7 wherein the biocatalyst comprises cells of Escherichia coli.

9. The process of claim 7 wherein the biocatalyst comprises glutaraldehyde (GA) treated cells of Escherichia coli.

10. The process of claim 7 wherein the biocatalyst comprises pure enzymes isolated from the bacterial cells.

11. The process of claim 10 wherein the biocatalyst comprises thymidine phosphorylase, uridine phosphorylase, or mixtures thereof, and purine nucleoside phosphorylase.

12. The process of claim 1 further comprising reacting the at least one purine base with the at least one donor of a carbohydrate moiety in the presence of an effective amount of a buffer.

13. The process of claim 12 wherein the buffer is potassium phosphate or sodium phosphate.

14. A biocatalytic process for preparing purine aminodeoxy N⁹-β-D-nucleosides comprising reacting at least one purine base with at least one selected from the group consisting of 3′-amino-3′-deoxyuridine, 3′-amino-3′-deoxythymidine and 2′-amino-2′-deoxyuridine in the presence of a biocatalyst wherein the purine base undergoes regio- and stereo-selective transglycosylation.

15. The process of claim 14 wherein the biocatalyst comprises bacterial cells or glutaraldehyde (GA) treated bacterial cells.

16. The process of claim 14 wherein the biocatalyst comprises thymidine phosphorylase, uridine phosphorylase, or mixtures thereof, and purine nucleoside phosphorylase.
